SEQUENCE LISTING (3 sequences)

SEQ ID NO: 1 - protein, 28 aa, Homo sapiens
YADAIFTSYRKVLGGLSARKLLQDIMSR

SEQ ID NO: 2 - DNA, 18 bp, Artificial Sequence
  Synthetic
caacagcagcaacagcaa

SEQ ID NO: 3 - protein, 12 aa, Homo sapiens
CTAAPLKPAKSC

(19) United States
(12) Patent Application Publication
(10) Pub. No.: US 2010/0158923 A1
(43) Pub. Date: Jun. 24, 2010

(54) REDUCING POLYGLUTAMINE-BASED AGGREGATION

(75) Inventors: Richard I. Morimoto, Evanston, IL (US); James F. Morley, Evanston, IL (US)

Correspondence Address: ELMORE PATENT LAW GROUP, PC, 515 Groton Road, Unit 1R, Westford, MA, 01886, US

(73) Assignee: Northwestern University

(21) Appl. No.: 12/689,494

(22) Filed: Jan. 19, 2010

Related U.S. Application Data
(63) Continuation of application No. 10/891,929, filed on Jul. 14, 2004, now abandoned.
(60) Provisional application No. 60/487,345, filed on Jul. 14, 2003.

(51) Int. Cl. A61K 38/31; A61K 39/395; A61K 38/27; A61K 38/05; A61K 38/08; A61P 25/14; A61P 25/00; G01N 33/53; C40B 30/04; C12N 5/10

(52) U.S. Cl. 424/158.1; 424/172.1; 514/11; 514/12; 514/19; 435/7.1; 506/9; 435/325

(57) ABSTRACT

The disclosure features, inter alia, methods for treating or preventing neurodegenerative disorders and disorders that caused at least in part by polyglutamine aggregation. The method can include reducing activity of the IGF-1/GH axis in a subject. One exemplary neurodegenerative disorder that is also caused at least in part by polyglutamine aggregation is Huntington's disease.

### CROSS-REFERENCE TO RELATED APPLICATIONS

This application is a continuation of U.S. application Ser. No. 10/891,929 filed on Jul. 14, 2004, which claims the benefit of U.S. Application Ser. No. 60/487,345 filed on Jul. 14, 2003. The entire teachings of the above applications are incorporated by reference herein.

### FEDERALLY SPONSORED RESEARCH OR DEVELOPMENT

The U.S. Government may have certain rights in this invention pursuant to Grant No. NIGMS38109, awarded by the National Institutes of Health.

### BACKGROUND

The folding and maintenance of proteins in their native conformation is essential to cellular function. Disruption of protein-folding homeostasis, leading to the appearance of protein aggregates, is associated with an increasing number of human diseases (1, 2). A prototypical class of these disorders, composed of at least eight progressive neurodegenerative diseases including Huntington's disease, is associated with genes containing (CAG)ₙ trinucleotide repeats encoding polyglutamine (polyQ) tracts in otherwise unrelated proteins (3, 4). Expression of expanded polyQ, with or without flanking sequences, is sufficient to recapitulate the pathological features of the diseases in multiple model systems, supporting a central role for the expansion in the etiology of these disorders (5-7).

Molecular genetic studies have established that huntingtin alleles from normal chromosomes contain fewer than 30-34 CAG repeats, whereas those from affected chromosomes contain more than 35-40 repeats (8, 9). These observations have led to the suggestion of a 35-40-residue threshold at which the disease gene products are converted to a proteotoxic state. Analysis of patient databases has established a strong inverse correlation between repeat length and age of onset (9-11). However, both this correlation and disease penetrance are much weaker for repeats of 42 or fewer residues (12), suggesting that substantial variation in the behavior of polyQ-containing proteins can exist at near-threshold repeat lengths, which influences the course of pathology.

### SUMMARY

This disclosure features, inter alia, methods for treating or preventing neurodegenerative disorders and disorders that are caused at least in part by polyglutamine aggregation. One exemplary neurodegenerative disorder that is caused at least in part by polyglutamine aggregation is Huntington's disease. Clinically, Huntington's disease is characterized by an involuntary choreiform movement disorder, psychiatric and behavioral changes and dementia. The age of onset is usually between the thirties and fifties, although juvenile and late onset cases of HD occur.

At the cellular level, Huntington's disease is characterized, at least in part, by protein aggregation in the cytoplasm and nucleus of neurons. Further examination of the protein aggregates revealed that the aggregates comprise ubiquitinated terminal fragments of Huntingtin. In human cells, ubiquitinated proteins or protein fragments are degraded by the proteasome system. There is accumulating evidence that the proteasome degradation system does not properly clear protein aggregates in diseases such as Huntington's Disease. The protein aggregates may themselves cause the proteasome to malfunction. See, e.g., Bence et al., Science 292: pp. 1552-1555 (2001). See also Waelter et al., Molecular Biology of the Cell 12: pp. 1393-1407 (2001).

In one aspect, the disclosure features a method of treating or preventing a neurodegenerative disorder in a subject. The method includes reducing activity of the IGF-1/GH axis in the subject. For example, the subject is a mammal, particularly a human. In one embodiment, the neurodegenerative disorder is caused at least in part by aggregation of a polyglutamine protein. Exemplary neurodegenerative disorders include: Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCA7), and Spinocerebellar Ataxia 12 (SCA12). In a particular embodiment, the neurodegenerative disorder is Huntington's disease.

In one embodiment, the method includes administering a composition that reduces IGF-1/GH axis activity. Typically, the composition is administered in an amount effective to reduce or prevent at least one symptom of the disorder (e.g., a clinical symptom) or in an amount effective to reduce or prevent polyglutamine-based aggregation. In one embodiment, the composition includes an agonist of an inhibitory component of the IGF-1/GH axis. For example, the inhibitory component of the IGF-1/GH axis is a somatostatin receptor (SST2 or SST5), a PTEN transcription factor, or a FOXO transcription factor (e.g., Forkhead). Exemplary agonists for inhibitory components include somatostatin, L-054,522, BIM-23244, BIM-23197, BIM-23268, octreotide, TT-232, butreotide, lanreotide, or vapreotide, as well as others described herein and that those that can be identified by the methods described herein.

In another embodiment, the composition includes an antagonist of an activator of the IGF-1/GH axis or a component that promotes or is required for an activity of the IGF-1/GH axis. For example, the component of the IGF-1/GH axis is GH, GHRH, GHRH-R, GHS, GHS-R, GH-R, PI-3 kinase, PDK-1, or an AKT kinase. In one embodiment, the antagonist is a kinase inhibitor. In another embodiment, the antagonist is an antibody to a hormone (e.g., GH, GHS, or GHRH) or an antibody or other agent that binds to a cell surface receptor (e.g., GH-R, GHRH-R, or GHS-R). Functional antibody fragments can also be used. In one embodiment, the antagonist is a modified ligand of the cell surface receptor. For example, the antagonist is a modified growth hormone molecule that antagonizes GH-R, e.g., Pegvisomant.

An exemplary antagonist of GHS or the GHS-R is a modified peptide, e.g., [D-Lys³]-GHRP-6.

In another embodiment, the composition includes a compound that is a dopamine agonist that decreases GH production.

In one embodiment, the composition includes an agent described herein, e.g., listed in Table 2.

Generally, a compound in the composition that modulates GH/IGF-1 axis activity can be a small organic molecule (e.g., less than 7 kDa in molecular weight, e.g., 6, 5, 4, 3, 2, 1, or 0.5 kDa). The compound can also be a peptide, polypeptide, antibody, antibody fragment, peptidomimetic, peptoid, nucleic acid, or other chemical compound or a combination of any of these.

In one embodiment, the composition is administered at regular intervals (e.g., daily, weekly, biweekly, or monthly). In yet another embodiment, the composition is administered at regular intervals for at least two months (e.g., preferably, at least six or nine months or for at least one, two, five, ten, 20, 25, or 30 years).

In an embodiment, the method further includes, e.g., prior to the reducing the activity of the IGF-1/GH axis, identifying the subject as a subject having or predisposed to having the disorder.

In another aspect, the disclosure features a method of treating a subject. The method includes: identifying a mammalian subject as having or being disposed to having a disorder caused at least in part by aggregation of polyglutamine; and providing a treatment to the subject, wherein the treatment antagonizes activity of the IGF-1/GH axis in the subject. The treatment can be prophylactic or provided as a curative (e.g., after the onset of at least one symptom). For example, the subject is a mammal, particularly a human. In one embodiment, the neurodegenerative disorder is caused at least in part by aggregation of a polyglutamine protein. Exemplary neurodegenerative disorders include: Spinalbulbar Muscular Atrophy (SBMA or Kennedy's Disease) Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCA7), and Spinocerebellar Ataxia 12 (SCA12). In a particular embodiment, the neurodegenerative disorder is Huntington's disease.

In one embodiment, the treatment includes administering a composition that reduces IGF-1/GH axis activity.

Typically, the composition is administered in an amount effective to reduce or prevent at least one symptom of the disorder (e.g., a clinical symptom) or in an amount effective to reduce or prevent polyglutamine-based aggregation. The pharmaceutically “effective amount” for purposes herein is determined by such considerations as are known in the art. The amount is effective either to achieve improvement in at least one clinical signs and/or symptoms—including but not limited to decreased levels of polyglutamine aggregation (e.g., aggregation of huntingtin), or improvement or elimination of symptoms and other clinical endpoints—or to delay onset of or progression of signs or symptoms of disease, as are selected as appropriate clinical indicia. Cure is not required, nor is it required that improvement or delay, as above described, be achievable in a single dose.

In one embodiment, the treatment is sufficient to reduce levels of GH, levels of IGF-1, levels of IGF-1 receptor signalling in the subject by at least 30% (e.g., at least 50, 60, 70, or 80%) of a normal level for the chronological age of the adult subject, but not below detection. The reduction can include reducing the level to a resulting level that is less than 90, 80, 70, 60, 50, or 30% and/or greater than 70, 65, 60, 55, 50, 45, 40, or 15% of the initial level of the subject. In another example, partial reduction can include reducing a level to a resulting level that is less than 90, 80, 70, 60, 50, or 30% and/or greater than 70, 65, 60, 55, 50, 45, 40, or 15% of the average level among normal individuals having the same age and gender as the subject.

Treatment can be commenced at least prior to clinical onset of the disorder or provided at least at some point after clinical onset of the disorder or onset of at least one symptom (e.g., clinical symptom) of the disorder.

In one embodiment, the subject is an adult. Typically, the subject is an adult (e.g., a human adult having an age of at least 18, 21, 24, or 28 years) without defects in the GH/IGF-1 axis, and thus does not have acromegaly, diabetic retinopathy, or another disorder that is symptomatic of an abnormality of the GH/IGF-1 axis. In one embodiment, the adult is at least middle aged, e.g., at least 50, 60, 65, 70, 75, or 80 years of age.

In one embodiment, the composition includes an agonist of an inhibitory component of the IGF-1/GH axis. For example, the inhibitory component of the IGF-1/GH axis is a somatostatin receptor (SST2 or SST5), a PTEN transcription factor, or a FOXO transcription factor (e.g., Forkhead). Exemplary agonists for inhibitory components include somatostatin, L-054,522, BIM-23244, BIM-23197, BIM-23268, octreotide, TT-232, butreotide, lanreotide, or vapreotide, as well as others described herein and that those that can be identified by the methods described herein.

In another embodiment, the composition includes an antagonist of an activator of the IGF-1/GH axis or a component that promotes or is required for an activity of the IGF-1/GH axis. For example, the component of the IGF-1/GH axis is GH, GHRH, GHRH-R, GHS, GHS-R, GH-R, PI-3 kinase, PDK-1, or an AKT kinase. In one embodiment, the antagonist is a kinase inhibitor. In another embodiment, the antagonist is an antibody to a hormone (e.g., GH, GHS, or GHRH) or an antibody or other agent that binds to a cell surface receptor (e.g., GH-R, GHRH-R, or GHS-R). Functional antibody fragments can also be used. In one embodiment, the antagonist is a modified ligand of the cell surface receptor. For example, the antagonist is a modified growth hormone molecule that antagonizes GH-R, e.g., Pegvisomant.

An exemplary antagonist of GHS or the GHS-R is a modified peptide, e.g., [D-Lys³]-GHRP-6.

In another embodiment, the composition includes compound that is a dopamine agonist that decreases GH production.

In one embodiment, the composition includes an agent described herein, e.g., listed in Table 2.

Generally, a compound in the composition that modulates GH/IGF-1 axis activity can be a small organic molecule (e.g., less than 7 kDa in molecular weight, e.g., 6, 5, 4, 3, 2, 1, or 0.5 kDa). The compound can also be a peptide, polypeptide, antibody, antibody fragment, peptidomimetic, peptoid, nucleic acid, or other chemical compound or a combination of any of these.

In one embodiment, the composition is administered at regular intervals (e.g., daily, weekly, biweekly, or monthly). In yet another embodiment, the composition is administered at regular intervals for at least two months (e.g., preferably, at least six or nine months or for at least one, two, five, ten, 20, 25, or 30 years).

In one embodiment, the method further includes monitoring the subject, e.g., for a symptom of the disorder, e.g., for a neurological, anatomical, or biochemical symptom, before, during, and/or after the reducing the activity of the GH/IFG-1 axis. In one embodiment, the monitoring includes imaging neuronal tissue (e.g., at least a part of the brain) of the subject. Images can be evaluated for indications of neuronal cell death, brain lesions, anomalous white matter, and protein aggregates.

In one embodiment, the monitoring includes a neurological exam (e.g., a cognitive exam, reflex test) or one or more subsections of the Unified Huntington's disease Rating Scale (UHDRS).

In one embodiment, the subject is monitored for a parameter of the IGF-1/GH axis.

In one embodiment, the identifying includes evaluating the identity of at least one nucleotide of a gene (or mRNA) of a subject. In an embodiment, the identifying includes evaluating a genetic relative of a subject for a symptom of a neurodegenerative disorder.

In one embodiment, the method includes: a) evaluating an indicator of GH/IGF-1 axis activity in the subject; and b) administering, to the subject, a regimen of doses of a compound that alters activity of a GH/IGF-1 axis component. The regimen is a function of the indicator and can be effective to maintain detectable, subnormal levels of IGF-1 in the subject with respect to age. Exemplary indicators of GH/IGF-1 axis activity include a parameter (e.g., concentration) that is a function of circulating hormone levels (e.g., GH or IGF-1), intracellular signaling, pituitary or hypothalamus physiology, and so forth. In a related method, an age-associated parameter or a parameter that is a function of caloric restriction is evaluated.

In another aspect, the disclosure features a kit that includes an active agent that antagonizes the IGF-1/GH pathway and instructions for or administering the agent to treat or prevent a neurodegenerative disorder or a disorder caused at least in part by polyglutamine aggregation. The agent can be an agent described herein.

In another aspect, the disclosure features a labeled container that includes a pharmaceutical composition that includes an active agent that antagonizes the IGF-1/GH pathway, wherein the container includes information for administering the composition to treat or prevent a neurodegenerative disorder or a disorder caused at least in part by polyglutamine aggregation.

Other exemplary methods for treating or preventing a neurodegenerative disorder including using an agent that increase hsf (heat shock factor) activity or expression in cells of a subject, e.g., neuronal cells of the subject. The method can include using a viral vector that infects neuronal cells to deliver a nucleic acid encoding hsf, e.g., hsf1. For example, US 20020107213 provides exemplary gene therapy systems for delivering a nucleic acid to a neuronal cell, e.g., glial cells or neurons.

The disclosure also provides a method for increasing HSF activity by modulating the IGF-1/GH axis, e.g., reducing activity of the IGF-1/GH axis in a cell or in cells of a subject. A subject can be provided a therapy that reduces axis activity in order to treat, prevent, or ameliorate at least one symptom of a disorder characterized by being improved by increased HSF activity.

Screening Methods

In another aspect, the disclosure features a method of evaluating a compound for ability to modulate polyglutamine aggregation in a cell. The method includes: a) providing a test compound; b) contacting the test compound to a GH/IGF-1 axis component in vitro; c) evaluating interaction between the test compound and the growth hormone/IGF-1 axis component; d) contacting the test compound to a cell; and e) evaluating polyglutamine aggregation in or around the cell or evaluating the cell for a cellular symptom of polyglutamine aggregation.

A related method includes: a) providing a library of compounds, the library including multiple compounds; b) contacting each compound of the library to a GH/IGF-1 axis component in vitro; c) evaluating interaction between each compound and the GH/IGF-1 axis component; d) selecting a subset of compounds from the library based on the evaluated interactions; and e) for each compound of the subset, contacting the compound to a cell, and evaluating polyglutamine aggregation in or around the cell or evaluating the cell for a cellular symptom of polyglutamine aggregation.

In one embodiment, the cell is a eukaryotic (e.g., mammalian) cell. For example, the cell expresses a heterologous protein that includes a polyglutamine repeat that includes at least 35 glutamines (e.g., at least 45, 50, 60, 70, or 80 glutamines). In one embodiment, the heterologous protein can also include all or part of a human protein that is a polyglutamine disease protein. For example, the heterologous protein includes at least 50 amino acid of the amino acid sequence of exon 1 of the human Huntingtin protein. Homologues of such human proteins can also be used. In another embodiment, the cell expresses an endogenous protein that includes a polyglutamine repeat that includes at least 35 glutamines. For example, the heterologous protein includes a fluorophore (e.g., the protein is a fluorescent protein, e.g., GFP, YFP, etc.).

In one embodiment, the cellular symptom of polyglutamine aggregation includes expression and/or subcellular localization of a heat shock protein. For example, the evaluating includes photobleaching and evaluating recovery of fluorescence after photobleaching.

The method can also include evaluating a parameter of the cell, in addition to polyglutamine aggregation, for example, an age-associated parameter of a cell (e.g., a neuronal cell, a fibroblast, an osteoblast, a skin cell, a blood cell, a transformed cell, a senescent cell, or any cultured cell) treated with the test compound. In one embodiment, the age-associated parameter includes one or more of: (i) lifespan of the cell or the organism; (ii) presence or abundance of a gene transcript or gene product in the cell or organism that has a biological age-dependent expression pattern; (iii) resistance of the cell or organism to stress; (iv) one or more metabolic parameters of the cell or organism; (v) proliferative capacity of the cell or a set of cells present in the organism; and (vi) physical appearance or behavior of the cell or organism. In another embodiment, the in vitro contacting is a cell-based assay or a cell-free assay.

Still another related method includes: a) providing a test compound; b) contacting the test compound to a GH/IGF-1 axis component in vitro; c) evaluating interaction between the test compound and the growth hormone/IGF-1 axis component; d) administering the test compound to a subject organism; and e) evaluating polyglutamine aggregation in the subject organism, a symptom of polyglutamine aggregation, or a neurological symptom.

Still another related method includes: a) providing a library of compounds, the library including multiple compounds; b) contacting each compound of the library to a GH/IGF-1 axis component in vitro; c) evaluating interaction between each compound and the GH/IGF-1 axis component; d) selecting a subset of compounds from the library based on the evaluated interactions; and e) for each compound of the subset, administering the compound to a subject organism, and evaluating the subject organism for polyglutamine aggregation, a symptom of polyglutamine aggregation, or a neurological symptom.

In one embodiment, the organism is an invertebrate organism. In another embodiment, the organism is a vertebrate organism, e.g., a non-human mammal. The organism can include cells containing a heterologous nucleic acid encoding a protein with a polyglutamine repeat region that includes at least 35 glutamines (e.g., at least 45, 50, 60, 70, or 80 glutamines). The heterologous nucleic acid can be a transgene or extrachromosomal element. The method can implemented using a cohort of organisms (e.g., non-human animals, e.g., mammals, e.g., rats, mice, primates, cows, pigs, and so forth). Statistics may be used to evaluate the cohort of organisms, e.g., to detect a statistically significant effect.

In one embodiment, the protein can also include all or part of a human protein that is a polyglutamine disease protein. For example, the protein includes at least 50 amino acid of the amino acid sequence of exon 1 of the human Huntingtin protein. Homologues of such human proteins can also be used. In another embodiment, the cell expresses an endogenous protein that includes a polyglutamine repeat that includes at least 35 glutamines. For example, the heterologous protein includes a fluorophore (e.g., the protein is a fluorescent protein, e.g., GFP, YFP, etc.).

The library can include at least 50, 10³, 10⁵, 10⁶, or 10⁸ compounds, e.g., between 10³ and 10⁷ compounds. The compounds can be less than 100 000, 60 000, or 30 000 Daltons. In another embodiment, the compounds can be less than 7000, 5000, or 3000 Daltons. In one embodiment, the library of compounds includes at least 50, 10³, 10⁵, 10⁶, or 10⁸ structurally related compounds, e.g., derivatives of a compound described herein. In one embodiment, the library includes a collection of naturally occurring compounds. In another embodiment, the library includes a collection of artificial compounds. The library can be a library of proteins, of nucleic acids (e.g., siRNAs), or precursors thereof (e.g., a library of nucleic acids that can be expressed to produce a library of proteins or that can be processed or transcribed to produce double-stranded RNAs (e.g., siRNAs)).

In one embodiment, the library includes multiple different spiropiperidine molecules (e.g. MK0677-like) or multiple different benzo-fused lactam molecules (e.g., L-739,943-like).

A further embodiment includes synthesizing a second library of compounds that include a set of features of a compound of the subset; and repeating the method. In another further embodiment the method includes formulating an identified compound as a pharmaceutical composition. The method can include other features described herein.

Screening Reagents

In another aspect, the disclosure features a non-human organism that includes a deficiency in a GH/IGF-1 axis component and a heterologous nucleic acid encoding a protein with a polyglutamine repeat region that includes at least 35 glutamines. The organism can be an invertebrate organism (e.g., a Drosophila or nematode) or a vertebrate organism (e.g., a non-human mammalian organism such as a rodent, e.g., a transgenic rodent). In one embodiment, the deficiency is caused by a genetic mutation. In another embodiment, the deficiency is caused by RNAi.

In another aspect, the disclosure features cultured cell preparation that includes: an engineered mammalian cell that expresses a protein with a polyglutamine repeat region of at least 35 glutamines; and medium containing a modulator (e.g., an agonist or antagonist) of the GH/IGF-1 axis (e.g., GH or IGF-1). The preparation can be used in a method for a test compound or a library of test compound. The method can include contacting a test compound to cells in the preparation; and evaluating the cells for aggregation of the protein with the polyglutamine repeats or a symptom of protein aggregation.

Information Management

In another aspect, the disclosure features a method for gathering genetic information, the method including: a) determining the identity of at least one nucleotide in gene encoding an IGF-1/GH axis component of a human subject; and b) creating a record which includes information about the identity of the nucleotide and information relating to a neurodegenerative disorder-related parameter from an evaluation of the subject.

A related method includes: a) determining the identity of at least one nucleotide in gene encoding an IGF-1/GH axis component for a plurality of subjects who have a neurodegenerative disorder or are associated with a neurodegenerative disorder; and b) evaluating the distribution of one or more nucleotide identities for a given position in the gene among or between subjects of the plurality.

The disclosure also features a computer-readable database that includes a plurality of records, each record including: a) a first field which includes information about one or more nucleotides from a gene encoding an IGF-1/GH axis component of a subject and; b) a second field which includes information about a phenotype of the subject, wherein the phenotype is associated with a neurodegenerative disorder, e.g., Huntington's disease. The information about the phenotype can include information about a biochemical parameter of the subject, anatomical parameter of the subject, or cognitive parameter of the subject. The information about the phenotype can include a diagnosis, e.g., a diagnosis of Huntington's disease.

In another aspect, the disclosure features a method of evaluating polyglutamine aggregation. The method includes: providing a cell that expresses a heterologous protein including a fluorescent protein and a polyglutamine repeat having a length of at least 35 glutamines; photobleaching the fluorescent protein in a cell; and evaluating distribution of fluorescence from the fluorescent protein in the cell. The fluorescent protein can be, e.g., a GFP or YFP. In another embodiment, the fluorescent protein is a protein that is coupled to fluorophore, e.g., rhodamine. The method can include maintaining the cell for an interval between the photobleaching and prior to the evaluating.

Growth hormone (GH) is a 22 kDa, 191 amino acid single chain peptide containing two disulfide bridges. In humans, GH is essential for linear growth of the infant, child, and adolescent and also plays an important role in the regulation of metabolism. In mammals, it is the primary hormone responsible for growth, and it accelerates metabolic processes such as lipolysis and protein synthesis. GH and many other hormones are part of a complex endocrine system, called the GH/insulin-like growth factor-1 axis (GH/IGF-1 axis).

GH secretion and circulating IGF-1 levels are regulated by the GH/IGF-1 axis. Included in the GH/IGF-1 axis are hormones from the hypothalamus and from elsewhere in the body, receptors on the anterior pituitary and peripheral tissues and organs, anterior pituitary somatotrophs that produce and secrete GH, and peripheral tissues that secrete IGF-1 in response to GH. FIG. 1 is a schematic of the GH/IGF-1 axis.

GH secretion occurs in a pulsatile manner due to the action of both positive and negative regulation originating from the hypothalamus. The hypothalamic peptide, GH releasing hormone (GHRH), and the endogenous GH secretagogue (GHS), ghrelin, are positive regulators of GH and act on the hypothalamus and/or anterior pituitary somatotrophs (cells that produce GH) to release GH. Human GHRH is a C-amidated 44 amino acid peptide. It is present and secreted from the hypothalamus. GHRH binds to specific GHRH receptors on the anterior pituitary thus causing GH release by the anterior pituitary somatotroph. Somatostatin, on the other hand, opposes the action of GHRH and ghrelin by blocking GH release. Somatostatin is a fourteen amino acid peptide that includes a cyclic loop bound by a disulfide bridge. Equally active synthetic versions of somatostatin can be in the reduced or linear state. Somatostatin is found in high concentrations in the hypothalamus, is produced by a large number of tissues, and participates in a wide array of biological functions, including decreasing GH release. Many neurotransmitters and neuropeptides are also involved in the control of GH secretion with both stimulatory and inhibitory effects, mostly via interaction with GHRH and somatostatin rather than direct interaction at the level of the pituitary.

As used herein, “activity of the GH/IGF-1 axis” refers to biological activities provided by the presence of GH and/or IGF-1 or by IGF-1 receptor signalling. Accordingly, “reducing activity of the GH/IGF-1 axis” refers to modulating one or more components such that one or more of the following is reduced, e.g., GH levels, IGF-1 levels, or IGF-1 receptor signalling. For example, in some instances, GH levels are maintained but its action is inhibited; thus IGF-1 levels are decreased without decreasing GH levels. In some instances, both GH and IGF-1 levels are decreased.

An “agonist of the GH/IGF-1 axis” increases one or more of the following GH levels, IGF-1 levels, or IGF-1 receptor signalling. For example, it can act by increasing the production, secretion, and/or activity of GH which subsequently causes a rise in IGF-1 production, secretion, or activity; or, it can increase IGF-1 levels, secretion or activity directly without affecting GH levels, e.g., by activating the GH receptor and/or the IGF-1 receptor. An agonist of the GH/IGF-1 axis may antagonize components which negatively regulate the GH/IGF-1 axis such as somatostatin or may promote components that positively regulate the GH/IGF-1 axis such as GHRH, e.g., by binding to GHRH or somatostatin, or by binding to GHRH receptor or somatostatin receptor(s).

An “antagonist of the GH/IGF-1 axis” decreases GH levels, IGF-1 levels, or IGF-1 receptor signalling. For example, it can act by decreasing the production, secretion, or activity of GH which subsequently causes a decrease in IGF-1 production, secretion, or activity; or, it can decrease IGF-1 levels, secretion, or activity directly without affecting GH levels, e.g., by inhibiting the GH receptor and/or the IGF-1 receptor. An antagonist of the axis GH/IGF-1 axis may negatively regulate an agonist of the GH/IGF-1 axis such as GHRH or may positively regulate an antagonist of the GH/IGF-1 axis such as somatostatin, e.g., by binding to GHRH or somatostatin, or by binding to GHRH receptor or somatostatin receptor(s).

Useful “antagonists of the GH/IGF-1 axis” include: a somatostatin agonist (e.g., an SST2 or SST5 agonist), a GHRH/GHRH-R antagonist, a GHS/GHS-R antagonist, a GH/GH-R antagonist, an IGF-1/IGF-1R antagonist, a PI3 kinase inhibitor, a PTEN agonist, a PDK-1 inhibitor, an AKT kinase inhibitor, or a Forkhead agonist. Some antagonists of the axis interfere with signaling events between axis components. Other antagonists, for example, interfere with expression, production, or secretion of an axis component. For example, double stranded RNA molecules (e.g., siRNA's) complementary to a nucleic acid encoding an axis component (particularly, GHRH, GHRH-R, GHS-R, GH, GH-R, IGF-1, IGF-1-R, PI3 kinase, PDK1, and AKT-1,2,3) can function as axis antagonists.

Generally, a receptor exists in an active (Ra) and an inactive (Ri) conformation. Drugs that affect the receptor can alter the ratio of Ra to Ri (Ra/Ri). For example, a full agonist increases the ratio of Ra/Ri and can cause a “maximal”, saturating effect.

A partial agonist, when bound to the receptor, gives a response that is lower than that elicited by a full agonist. Thus, the Ra/Ri for a partial agonist is less than for a full agonist. However, the potency of a partial agonist may be greater or less than that of the full agonist.

An inverse agonist produces an effect opposite to that elicited by an agonist when it binds to the receptor. In this instance there is a shift in the equilibrium to Ri (e.g., an increase in Ri/Ra or a decrease in Ra/Ri). A super agonist causes an ultra-high response when bound to receptors, typically as a result of a particularly strong efficacy. Efficacy can be a function of the ligand's “on-rate” and “off-rate” for binding to the receptor.

As used herein, a “polyglutamine region” of a protein is a region of the protein that includes at least 15 consecutive glutamine residues, and is at least 90 or 95% glutamine. Typically, the region is 100% glutamine and includes at least 30, 35, 40, 506, 70, 80, or 90 residues. Regions with greater than 35 glutamines are more prone to aggregation. Absent other factors, the propensity for aggregation increases with repeat length.

A subject with “normal” GH or IGF-1 levels is one who would return a normal result (with respect to age and gender) using the glucose tolerance test in which glucose is ingested and blood levels of GH are measured by radioimmunoassay (RIA) or polyclonal immunoassay. A subject with normal GH levels can typically have less than 1 ng/mL of GH within 1 to 2 hours of an oral glucose load. However, GH levels of a subject with excessive GH, as in one with acromegaly or diabetic retinopathy, will not decrease below 1 ng/mL after ingesting glucose. Because GH levels oscillate every twenty to thirty minutes and varies in level according to the time of day, stress level, exercise, etc., a standard means of determining if GH levels are excessive is to administer glucose. This approach normalizes GH and is less affected by the pulsatility of GH, age, gender, or other factors. Alternatively or as a confirmation, since IGF-1 levels are invariably increased in acromegalic individuals, IGF-1 levels can be measured and compared to age and gender matched normal controls. Normal levels can be within 0.5, 0.7, 0.8 or 1.0 standard deviations of the mean.

The term “an indicator of GH/IGF-1 axis activity” refers to a detectable property of the GH/IGF-1 axis that is indicative of activity of the axis. Exemplary properties include circulating GH concentration, circulating IGF-1 concentration, frequency of GH pulses, amplitude of GH pulses, GH concentration in response to glucose, IGF-1 receptor phosphorylation, and IGF-1 receptor substrate phosphorylation.

A “test compound” or “candidate compound” is any chemical compound, which may or may not affect the GH/IGF-1 axis. Exemplary test compounds include candidate proteins, peptides, peptidomimetics, peptoids, small molecules or other drugs. Exemplary small molecules have a molecular weight of less than 7000, 5000, 3000, or 2000 Daltons. Small molecules include, for example, benzolactams and spiroindanylpiperadines. A test compound can be soluble or insoluble in an aqueous solution. In one embodiment, an exemplary test compound is an agonist or antagonist of a compound described herein, e.g., a somatostatin agonist.

A “component of the GH/IGF-1 axis” includes a hormone that directly or indirectly regulates the axis (e.g., GH, IGF-1, ghrelin, GHRH, or somatostatin), a cell surface receptor (e.g., a hormone receptor, e.g., a GH receptor, a GHS receptor an IGF-1 receptor, a somatostatin receptor), intracellular signaling molecules (e.g., kinases, adaptor molecules, transcription factors), transcripts (encoding a protein component of the axis), effectors (e.g., proteins encoded by a gene induced by the axis), and so forth. Specific examples of intracellular signaling molecules include: PI(3) kinase, PTEN phosphatase, PI(3,4)P₂, and PI(3,4,5)P₃ phosphatidyl inositol kinases, AKT-1,2,3 serine/threonine kinase, and Forkhead transcription factors.

In one embodiment, the GH/IGH-1 axis component is no more than two components removed from GH, IGF-1, or the IGF-1 receptor. For example, an upstream component that is no more than two components removed may act through one or two intermediaries to modulate axis activity. In some embodiments, the GH/IGH-1 axis component is no more than one component removed (e.g., no more than one intermediary) between the component and GH, IGF-1, or the IGF-1 receptor. In another embodiment, the GH/IGH-1 axis component is no more than two components removed from PI(3) kinase, PTEN phosphatase, PI(3,4)P₂, and PI(3,4,5)P₃ phosphatidyl inositol kinases, AKT-1,2,3 serine/threonine kinase, or a Forkhead transcription factor.

Some Abbreviations: polyQ, polyglutamine; YFP, yellow fluorescent protein; FRAP, fluorescence recovery after photobleaching; RNAi, RNA interference; CBP, CREB-binding protein.

The details of one or more embodiments of the inventions are set forth in the description below. Other features, objects, and advantages of the inventions will be apparent from the description and the claims. The contents of all references, pending patent applications and published patents, cited throughout this application are hereby expressly incorporated by reference. The following applications are among those incorporated by reference, 60/487,345 and US 2004-0121407.

### BRIEF DESCRIPTION OF THE DRAWINGS

FIG. 1 is a schematic of the GH/IGF-1 axis.

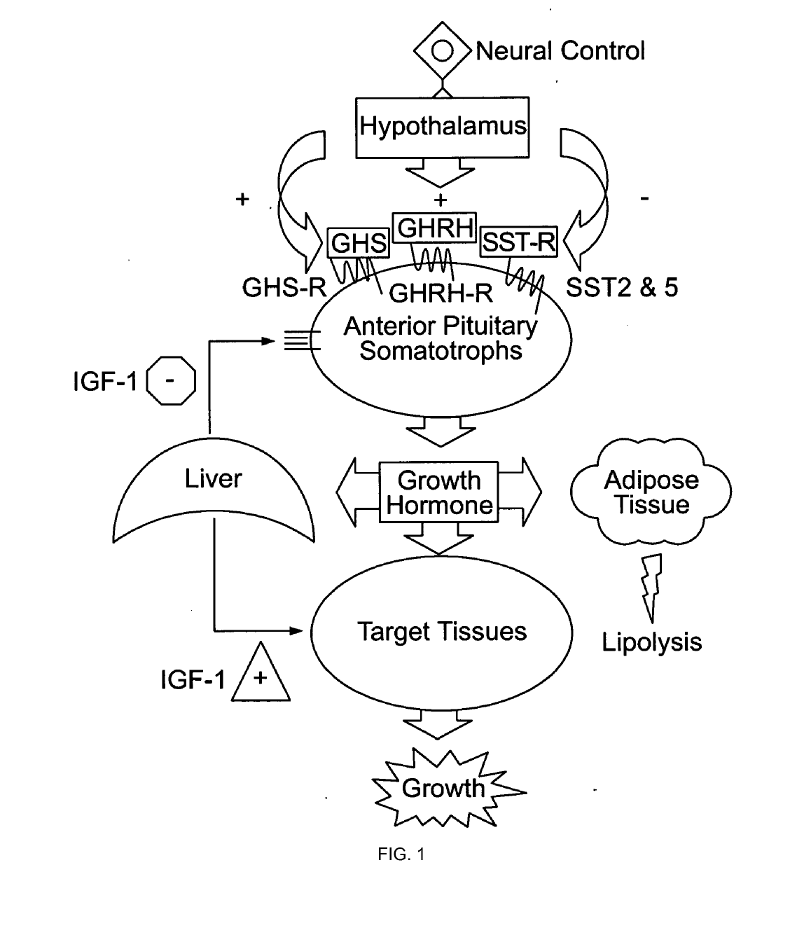

FIG. 2 is a schematic of some insulin/IGF-1 signalling components.

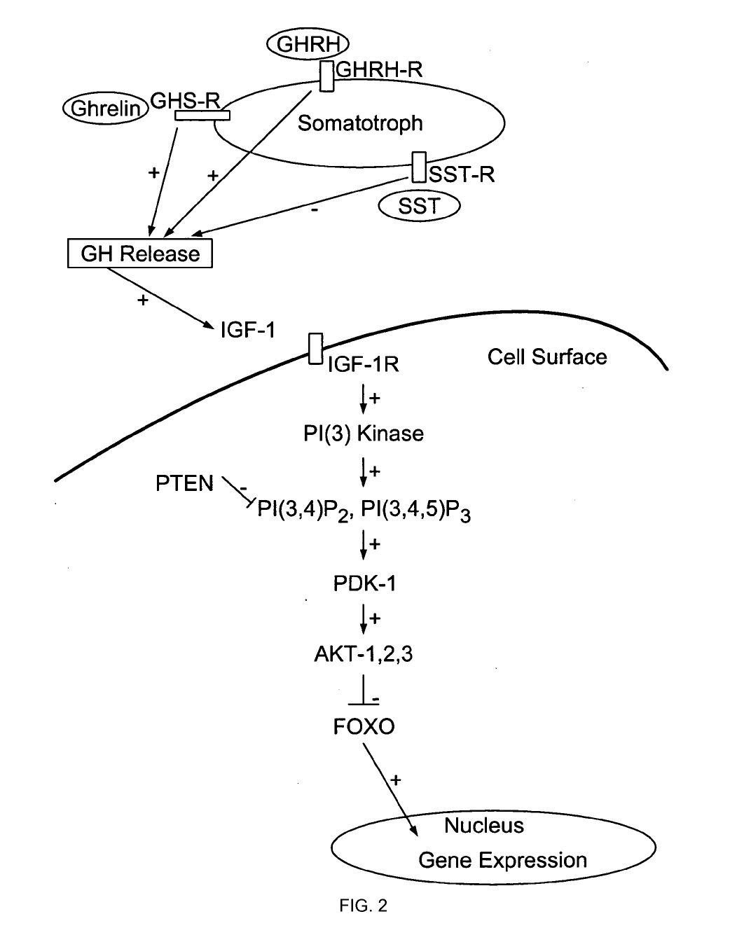

FIG. 3 is a schematic of a modification of L-054,522, an agonist, to provide an antagonist.

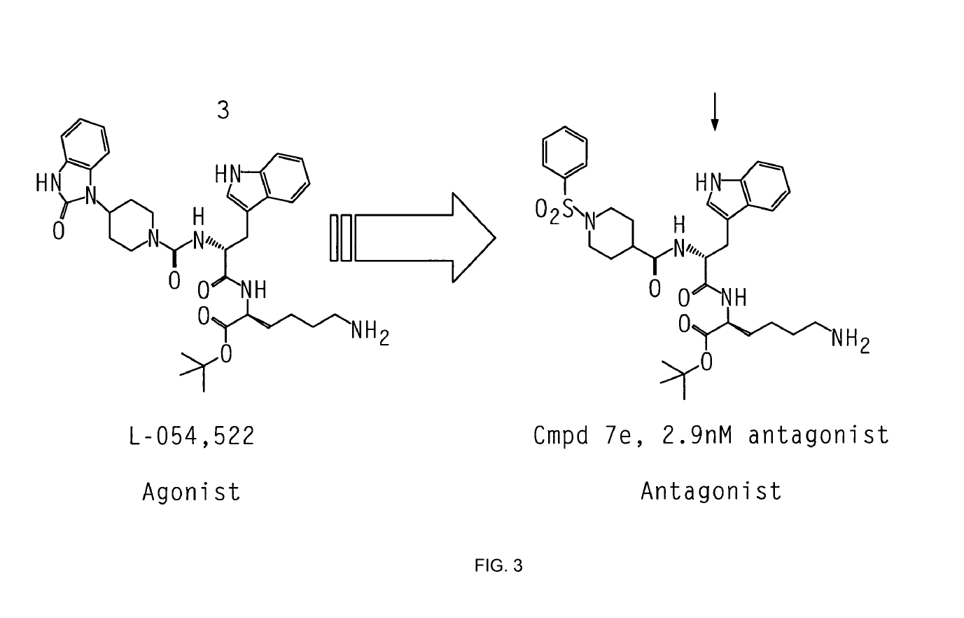

### DETAILED DESCRIPTION

This disclosure provides, inter alia, treatments and compositions that alter polyglutamine-based aggregation by antagonizing the GH/IGF-1 axis. In a particular reducing the activity of the axis by reducing activity of PI3 kinase in a model organism reduces polyglutamine-based aggregation of a reporter protein.

There are a number of disorders whose pathologies have been attributed, at least in part, to polyglutamine-based aggregation. These disorders include, for example, Huntington's disease, Spinalbulbar Muscular Atrophy (SBMA or Kennedy's Disease), Dentatorubropallidoluysian Atrophy (DRPLA), Spinocerebellar Ataxia 1 (SCA1), Spinocerebellar Ataxia 2 (SCA2), Machado-Joseph Disease (MJD; SCA3), Spinocerebellar Ataxia 6 (SCA6), Spinocerebellar Ataxia 7 (SCA7), and Spinocerebellar Ataxia 12 (SCA12).

Accordingly, reducing activity of the GH/IGF-1 axis can be used to treat or prevent such disorders. For example, a treatment that reduces activity of the axis may be given prophylactically, e.g., to an individual who is genetically or otherwise predisposed to such a disorder. The treatment may also be given to reduce at least one symptom of the disorder in an individual who manifests one or more symptoms of the disorder.

The GH/IGF-1 axis includes a number of components. Some of these components are inhibitory components since their biological activity antagonizes activity of the axis (e.g., antagonizes IGF-1 production). Other components promote or are otherwise required for activity of the axis. For example, these positively-acting components agonizes or otherwise contribute to IGF-1 production. The GH/IGF-1 axis also includes events downstream, e.g., subsequent, to detection of the IGF-1 signal. One downstream result may be the activation of effectors that antagonize or prevent polyglutamine aggregation. The methods described herein typically modulate polyglutamine aggregation without directly manipulating these effectors. For example, GH/IGF-1 axis activity is modulated such that more than a single isolated class of effectors (e.g., the class of chaperones) is affected. In other examples, however, one may also directly modulate such a class of effectors.

As the GH/IGF-1 axis includes a number of components, a variety of means can be used to modulate activity of the axis. For example, a compound that modulates activity of the axis can be administered to a subject, e.g., a human. In one embodiment, the compound being used can modulate GH/IGF-1 activity without having to traverse the blood-brain barrier. Since modulation of the GH/IGF-1 axis can cause systemic effects, reducing polyglutamine aggregation in cells of the brain may be possible by administering a compound that itself does need to traverse the blood-brain barrier.

There are a variety of methods that can be used to down regulate the GH/IGF-1 axis. For example, axis activity can be reduced by targeting a particular component of the axis. Depending on the component's function in the axis, it may be appropriate to inhibit its activity or to promote its activity. For example, axis activity can be reduced by agonizing an inhibitory component of the axis or antagonizing a component that promotes or is required for axis activity. Exemplary targets and the desired activity used against these targets to reduce axis activity are as follows:

TABLE 1
Pathway Targets

| Target | Desired Activity |
|---|---|
| SST2 & SST5 | Agonist |
| GHRH/GHRH-R | Antagonist |
| GHS/GHS-R | Antagonist |
| GH/GHR | Antagonist |
| IGF-1/IGF-1R | Antagonist |
| PI(3) kinase | Inhibitor |
| PTEN | Agonist |
| PDK-1 | Inhibitor |
| Akt-1, -2, -3 | Inhibitor |
| Forkhead | Agonist |

Of course, some molecules may fit more than one of the above classifications. Further exemplary details are provided below.

As seen in Table 1, these molecules include molecules which can target extracellular molecules: for example: a GH antagonist; a GH receptor antagonist; a GHRH antagonist; a GHRH receptor antagonist; IGF-1 receptor antagonist; IGF-1 antagonist; a somatostatin agonist; and; a somatostatin receptor agonist; as well as molecule that target intracellular molecules.

The net effect of reducing axis activity can be manifested, e.g., as reduced GH levels, reduced IGF-1 levels, reduced IGF-1 receptor signaling, reduced GHRH levels, reduced GHS levels, or increased somatostatin levels. In many cases, it is useful to reduce such levels below the norm, but to retain at least a detectable amount, e.g., a non-zero level. For example, partial reduction can include reducing a level to a resulting level that is less than 90, 80, 70, 60, 50, or 30% and/or greater 70, 65, 60, 55, 50, 45, 40, or 15% of the initial level of a subject. In another example, partial reduction can include reducing a level to a resulting level that is less than 90, 80, 70, 60, 50, or 30% and/or greater than 70, 65, 60, 55, 50, 45, 40, or 15% of the average level among normal individuals having the same age and gender as the subject.

Typically, the subject is an adult (e.g., a human adult having an age of at least 18, 21, 24, or 28 years) without defects in the GH/IGF-1 axis, and thus does not have acromegaly, diabetic retinopathy, or another disorder which is symptomatic of an abnormality of the GH/IGF-1 axis. Acromegaly is a disorder of excessive GH production which stimulates excessive IGF-1 production. A glucose tolerance test can be used determine if a person has excessive GH. GH in a normal person decreases to less than 1 or 2 ng/mL after ingestion of sugar whereas in the acromegalic person, GH does not decrease below 1 or 2 ng/mL.

Somatostatin Agonists

Somatostatin and somatostatin agonists can be used to downregulate the GH/IGF-1 axis and thereby reduce polyglutamine aggregation. As used herein a “somatostatin agonist” is a compound that has at least one biological function of somatostatin and that can alter regulation of the GH/IGH-1 axis. The recombinant form of somatostatin as well as somatostatin octapeptides have been used to treat acromegaly. One useful somatostatin agonist is L-054,522. See, e.g., Pasternak et al. (1999) Bioorganic & Medicinal Chemistry Letters which also provides L-054,522 related compounds with improved bioavailability; and Yang et al. (1998) Proc Nat Acad USA 95:10836. L-054,522 binds to human SST2 with an apparent K_{d} of 0.01 nM and is highly selective. One exemplary L-054,522 compound has the following structure:

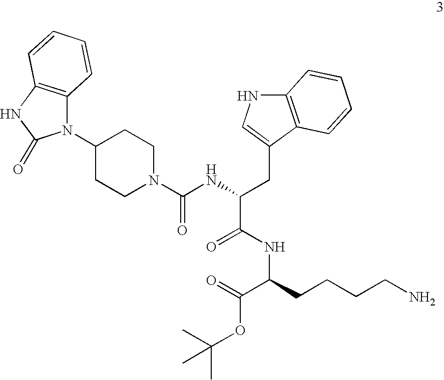

Other useful somatostatin agonists include BIM-23244, BIM-23197, BIM-23268, octreotide, TT-232, butreotide, lanreotide, and vapreotide. Octreotide and lanreotide are currently approved for treatment of acromegaly. These bind the receptors on the anterior pituitary gland and function to lower the production and secretion of GH.

Somatostatin is a hypothalamic factor that, among other biological functions, suppresses the secretion of GH from the anterior pituitary. It is produced by a large number of tissues. Due to its rapid degradation and clearance, somatostatin is not a truly circulating hormone. It is produced locally to its site of function, presumably to prevent inappropriate activation of receptors in tissues throughout the body. In developing drugs that mimic somatostatin, a key goal is to increase its stability thus extending its circulating half-life. In one embodiment, a somatostatin analog has local tissue specificity. For example, it may bind a subset of the five distinct receptor subtypes that bind to somatostatin, particularly the SST2 or SST5 receptors.

GH Antagonists

GH antagonists can be used to downregulate the GH/IGF-1 axis and thereby reduce polyglutamine aggregation. GH antagonists include molecules which antagonize production (e.g., synthesis or secretion) of GH. GH antagonists include a naturally occurring antagonist—somatostatin—and pharmaceuticals. Exemplary pharmaceuticals include those used to treat acromegaly (a disorder of excessive GH) by antagonizing GH are the somatostatin agonists (see above) and dopamine agonists, such as bromocriptine (Parlodel), pergolide (Permax), and cabergoline (Dostinex).

Dopamine Agonists. Dopamine agonists, such as bromocriptine, pergolide, and cabergoline, are synthetic compounds that act like the naturally occurring compound dopamine to reduce GH secretion. Thus, these agents can be used to alter polyglutamine aggregation.

GH Receptor Antagonists

GH receptor antagonists include molecules that antagonize the function of the GH receptor, for example, by preventing binding of GH or GH receptor dimerization. GH receptor antagonists can be used to alter polyglutamine aggregation.

Pegvisomant. An example of a GH receptor antagonist is Pegvisomant. Pegvisomant (Somavert) is a modified human GH in which nine amino acids have been replaced thus preventing receptor dimerization. Normally a single GH molecule binds to two GH receptor molecules to allow their dimerization. These amino acid changes at the dual receptor binding site of human GH allow Pegvisomant to bind more strongly to a single receptor molecule with inhibition of binding to the second receptor molecule, thus preventing dimerization of the GH receptor. Polyethylene glycol polymers on Pegvisomant decrease its rate of clearance, reduce its immunogenicity, and enhance its bioactivity. Pegvisomant is one available treatment for acromegaly. It has been observed that Pegvisomant administered subcutaneously causes a dose dependent reduction in IGF-1 levels.

GHS/GHS-R Antagonists

Antagonists of growth hormone secretagogues (GHS) and GHS receptors can be used to downregulate the GH/IGF-1 axis and thereby reduce polyglutamine aggregation.

An exemplary GHS antagonist is [D-Lys3]-GHRP-6, antagonist for Growth Hormone Releasing Peptide 6 (see also His-D-Trp-D-Lys-Trp-D-Phe-Lys-NH2; Sigma-Aldrich Product No. G4535). Other antagonists include compounds that interact with the GHS-receptor and an endogenous GHS, e.g., ghrelin. For example, antibodies to ghrelin can be used as antagonists. See, e.g., Nakazato et al. (2001) Nature 409:194. Similarly, ligands that bind to GHS receptors, e.g., antibody ligands, can be used to antagonize the axis and reduce polyglutamine aggregation.

GHRH Antagonists

GHRH is a peptide present in the hypothalamus which causes GH release from the anterior pituitary by interacting with specific GHRH receptors. A “GHRH antagonist” antagonizes the function of GHRH, e.g., by preventing or competing for receptor binding. GHRH antagonists decrease secretion of GH by the anterior pituitary somatotroph. An example of a GHRH antagonist is [N-acetyl-Tyr¹,D-Arg²] GHRH¹⁻²⁹NH₂, herein referred to as the “standard GHRH antagonist.” The standard GHRH antagonist, which is a modified version of the first 29 residues of GHRH (the shortest fragment of GHRH that possesses GH-releasing capability and binding properties) lowers spontaneous GH secretion and inhibits human GH secretory response to exogenous GHRH (Nargund et al., Journal of Medicinal Chemistry 41:3103-3127, 1998; Dimaraki et al., Proceedings of the 83^{rd} Meeting of the Endocrine Society, p. 97, Abstract 0R22-3).

The sequence of the first 29 residues of GHRH that still possesses GH-releasing capability and binding properties, thus referred to as the bioactive core of GHRH, is as follows:

(SEQ ID NO: 1)

Tyr¹-Ala-Asp-Ala-Ile-Phe-Thr-Ans-Ser-Tyr-Arg-Lys-
Val-Leu-Gly-Gln-Leu-Ser-Ala-Arg-Lys-Leu-Leu-Gln-
Asp-Ile-Met-Ser-Arg²⁹

The standard GHRH antagonist and many other antagonists have a D-Arg in the second position which confers its antagonist activity. More potent GHRH inhibitors can be constructed with certain hydrophobic and helix-stabilizing amino acid substitutions, such as para-chlorophenylalanine (Phe(4-Cl)) in position six, α-aminobutyric acid (Abu) in position fifteen, and norleucine (Nle) in position 27, combined with a hydrophobic N-terminal acyl moiety, such as iso-butyryl (Ibu-), phenylacetyl (PhAc-) or 1-naphthylacetyl (Nac-) (Schally and Varga, Trends Endocrinol. Metab., 10:382-391, 1999; Zarandi et al., Proc. Natl. Acad. Sci. USA 91:12298-12302, 1994; U.S. Pat. No. 6,057,422). Replacement of the Arg residue in position 29 with agmatine (Agm), combined with the N-terminal acylation of the analogs contributes to enzymatic stability and protracted antagonistic activity in vivo as compared to the standard GHRH antagonist. Small molecule mimetics of these antagonists can also be produced based on the 3-dimensional structures described above. In addition to inhibiting GH release, GHRH antagonists may indirectly decrease pituitary production of GH and of GH-mediated hepatic synthesis of IGF-1.

Accordingly, a GHRH antagonist can be used to attenuate activity of the GH/IGF-1 axis and thereby reduce polyglutamine aggregation.

Agonist-Based Screening

In one aspect, the disclosure features a method of identifying an antagonist of the GH/IGF-1 axis. The method is based upon information about agonists of the axis.

In this method, the agonist serves as a starting point for a screen to identify chemically and structurally related compounds that may inhibit the axis, in particular compounds that antagonize rather than agonize the axis, and thereby reduce polyglutamine-based aggregation.

The method takes advantage of the fact that the agonist interacts with a component of the axis. Modification or similarity to the agonist may retain some physical aspects of the interaction with this component but may provide new properties that result in the opposite functional effect. For example, it is known that a dimeric ligand that agonizes a cell surface receptor, may antagonize it in monomeric form. Although the monomer may bind more poorly than the dimer, modification of the monomer to generate an additional binding interface may produce an effective antagonist.

A variety of processes can be used to implement the above method. These processes can be used in conjunction with a screening method described herein.

Chemical Libraries. In one example, combinatorial chemical libraries can be produced that sample chemical compounds that are structurally or chemically related. For example, a scaffold is selected based on information about the known agonist. Then various positions on the scaffold are modified in combination to produce a large number of different compounds. The diversity of particular positions can be precisely controlled.

Methods for producing chemical libraries are well known. See, for example, Cox et al. (2000) Prog Med Chem 37:83; Sternson (2001) Org Lett 3(26):4239-42; Tam et al. (1998) J. Am Chem. Soc. 120:8565; 1: Floyd et al. (1999) Prog Med Chem. 36:91-168; Rohrer et al. (1998) Science, 282(5389):737-40; Komarov et al. (1999) Science, 285(5434):1733-7; Mayer et al. (1999) Science, 286(5441):971-4.

Members of a chemical library can be tagged. In such libraries, the identity and composition of each member of the library is uniquely specified by the label or “tag” which is physically associated with it and hence the compositions of those members that bind to a given target or that have a particular activity are specified directly (see, e.g., Ohlmeyer et al., 1993, Proc. Natl. Acad. Sci. USA 90:10922-10926; Brenner et al., 1992, Proc. Natl. Acad. Sci. USA 89:5381-5383; Lerner et al., PCT Publication No. WO 93/20242). In other examples of such libraries, the library members are created by step wise synthesis protocols accompanied by complex record keeping, complex mixtures are screened, and deconvolution methods are used to elucidate which individual members were in the sets that had activity (e.g., binding or biological activity), and hence which synthesis steps produced the members and the composition of individual members (see, e.g., Erb et al., 1994, Proc. Natl. Acad. Sci. USA 91:11422-11426).

Structure-Activity Relationships and Structure-Based Design. It is also possible to use structure-activity relationships (SAR) and structure-based design principles to produce an agonist from an antagonist. SARs provide information about the activity of related compounds in at least one relevant assay. Correlations are made between structural features of a compound of interest and an activity. For example, it may be possible by evaluating SARs for a family of compounds related to a GH/IGF-1 axis agonist to identify one or more structural features required for the agonist's activity. A library of compounds can then be produced that vary these features. In a related example, features required for agonist activity, but not for binding to the component of the axis that is the target of the agonist can be varied.

Structure-based design can include determining a structural model of the physical interaction of a GH/IGF-1 axis agonist and its target. The structural model can indicate how an antagonist of the target can be engineered.

Both the SAR and the structure-based design approach can be used to identify a pharmacophore. Pharmacophores are a highly valuable and useful concept in drug discovery and drug-lead optimization. A pharmacophore is defined as a distinct three dimensional (3D) arrangement of chemical groups essential for biological activity. Since a pharmaceutically active molecule must interact with one or more molecular structures within the body of the subject in order to be effective, and the desired functional properties of the molecule are derived from these interactions, each active compound must contain a distinct arrangement of chemical groups which enable this interaction to occur. The chemical groups, commonly termed descriptor centers, can be represented by (a) an atom or group of atoms; (b) pseudo-atoms, for example a center of a ring, or the center of mass of a molecule; (c) vectors, for example atomic pairs, electron lone pair directions, or the normal to a plane. Once formulated a pharmacophore can be used to search a database of chemical compound, e.g., for those having a structure compatible with the pharmacophore. See, for example, U.S. Pat. No. 6,343,257; Y. C. Martin, 3D Database Searching in Drug Design, J. Med. Chem. 35, 2145 (1992); and A. C. Good and J. S. Mason, Three Dimensional Structure Database Searches, Reviews in Comp. Chem. 7, 67 (1996). Database search queries can be based not only on chemical property information but also on precise geometric information.

Computer-based approaches can use database searching to find matching templates; Y. C. Martin, Database searching in drug design, J. Medicinal Chemistry, vol. 35, pp 2145-54 (1992), which is herein incorporated by reference. Existing methods for searching 2-D and 3-D databases of compounds are applicable. Lederle of American Cyanamid (Pearl River, N.Y.) has pioneered molecular shape-searching, 3D searching and trend-vectors of databases. Commercial vendors and other research groups also provide searching capabilities (MACSS-3D, Molecular Design Ltd. (San Leandro, Calif.); CAVEAT, Lauri, G. et al., University of California (Berkeley, Calif.); CHEM-X, Chemical Design, Inc. (Mahwah, N.J.)). Software for these searches can be used to analyze databases of potential drug compounds indexed by their significant chemical and geometric structure (e.g., the Standard Drugs File (Derwent Publications Ltd., London, England), the Bielstein database (Bielstein Information, Frankfurt, Germany or Chicago), and the Chemical Registry database (CAS, Columbus, Ohio)).

Once a compound is identified that matches the pharmacophore, it can be tested for activity, e.g., for binding to a component of the GH/IGF-1 axis and/or for a biological activity, e.g., modulation of the axis, e.g., reduce activity of the axis. See, e.g., “Screening Methods” below.

The following are examples of known agonists of the GH/IGF-1 axis. Each of these agonists can serve as a base compound for identifying an antagonist of the axis.

GHRH Agonists

Agonists of GHRH serve to increase GH. Examples of known GHRH agonists are GHRH¹⁻⁴⁴NH₂ and GHRH¹⁻²⁹NH₂. One type of structural library that can be based on these agonists are peptide libraries in which subregions of the 29 amino acid peptide sequence are varied, and tested for modulation of the IGF-1 axis.

GHS Agonists

Another class of molecules that can be modified to find an agent that down regulates the pathway is the class of GHS agonists. At least some of these agonists have been used to treat patients with a GH deficiency. Exemplary GHS agonists include, ghrelin, GHS-6, MK-0677 (L-163,191) and L-739,943. The endogenous GH secretagogue is ghrelin. MK-0677 is a spiroindanylpiperadine with potent GH-releasing effects when administered orally and parenterally (Patchett (1995) Proc Nat Acad USA 92:7001). Its structure is as follows:

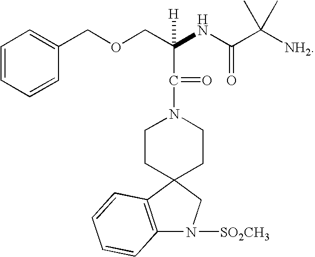

L-739,943, a potent, orally bioavailable benzolactam GH secretagogue, is obtained from zwitterionic L-692,429 through modification of its amino acid side chain and replacement of the acidic 2′-tetrazole with the neutral and potency enhancing 2′-(N-methylaminocarbonylamino)methyl substituent. (De Vita et al., J Med Chem 41:1716-28, 1998). Other GH agonists include penta-, hex-, and heptapeptide analogs that specifically stimulated GH secretion from the anterior pituitary gland in a dose-dependent manner in vitro and in vivo. These include Leu- and Met-Enkephalin, GHRP-1 to GHRP-6, and hexarelin (Root and Root (2002), supra).

Other general agonists of GH action can also be used as a basis for identifying an axis antagonist. For example, arginine is a potent cholinergic agonist that has been successful in stimulating GH secretion even in the elderly in whom many GH agonists have not been as successful. Arginine analogs and pro-drugs can also be used as starting points for identifying an antagonist of the pathway. Still other agonists include SM-130686, an oxindole derivative (available, e.g., from Sumitomo), NN703 and hexarelin. Similarly, it is possible to use selective antagonists of somatostatin receptors, e.g., SST2, to develop somatostatin receptor agonists. Exemplary somatostatin receptor antagonists include BIM-23454 and BIM-23627 (Biomeasure).

Various libraries of compounds can be designed based on these compounds and other compounds with similar properties. The libraries can be screened to identify compounds that downregulate the GH/IGF-1 axis and modulate polyglutamine aggregation.

Screening Assays

A test compound can be evaluated for its effect on the GH/IGF-1 axis or for its ability to interact with a GH/IGF-1 component. Compounds that have an effect on the axis (e.g., reduce axis activity) can be evaluated to determine if they reduce polyglutamine aggregation, e.g., in a cell or organism, or to determine if they reduce at least one symptom of a neurodegenerative disorder. Methods include in vitro and/or in vivo assays. Interactions include, for example, binding a target component, altering a covalent bond in a target component, or altering a biological or physiological function of a target compound (e.g., altering production, stability, or degradation of a target component). A test compound that modulates the GH/IGF-1 axis (e.g., reduces axis activity) can be prepared as a pharmaceutical composition (see below) and administered to a subject.

The test compounds can be obtained, for example, as described above (e.g., based on information about an agonist) or using any of the numerous combinatorial library method. Some exemplary libraries include: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. These approaches can be used, for example, to produce peptide, non-peptide oligomer or small molecule libraries of compounds (see, e.g., Lam (1997) Anticancer Drug Des. 12:145).

A biological library includes polymers that can be encoded by nucleic acid. Such encoded polymers include polypeptides and functional nucleic acids (such as nucleic acid aptamers (DNA, RNA), double stranded RNAs (e.g., RNAi), ribozymes, and so forth). The biological libraries and non-biological libraries can be used to generate peptide libraries.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.). In many cases, a high throughput screening approach to a library of test compounds includes one or more assays, e.g., a combination of assays. Information from each assay can be stored in a database, e.g., to identify candidate compounds that can serve as leads for optimized or improved compounds, and to identify SARs.

Cell-Based Assays. In one embodiment, a cell-based assay is used to evaluate a test compound. The cell, for example, can be of mammalian origin, (e.g., from a human, a mouse, rat, primate, or other non-human), or of non-mammalian origin (e.g., Xenopus, zebrafish, or an invertebrate such as a fly or nematode). In some cases, the cell can be obtained from a transgenic organism, e.g., an organism which includes a heterologous GH/IGF-1 axis component, (e.g., from a mammal, e.g., a human).

In one example, a cell which expresses a GH/IGF-1 axis protein or polypeptide or biologically active portion thereof is contacted with a test compound, and the ability of the test compound to modulate the GH/IGF-1 axis is determined. Determining the ability of the test compound to modulate the GH/IGF-1 axis can be accomplished by monitoring, for example, GH and/or IGF-1 levels, e.g., by radioimmunoassay. For example, the assay can include evaluate GH or IGF-1 synthesis and release.

See Example 1, below which describes an assay using cultured pituitary cells. It is also possible to monitor an intracellular component of the GH/IGF-1 axis, e.g., abundance, activity or post-translational modification state of a PI(3)Kinase, a phosphatase (e.g., PTEN), a phosphoinositol kinase; or a serine-threonine kinase (e.g., an AKT kinase). Changes in post-translational modification can be monitored using modification specific antibodies, changes in electrophoretic mobility, and mass spectroscopy, for example.

Another exemplary cellular assay includes contacting a hormone responsive cell with a hormone (e.g., somatostatin, GH or IGF-1) in the presence of the test compound and evaluating a parameter (e.g., a qualitative or quantitative property) of the cell (e.g., expression of one or a profile of genes, abundance of one or more proteins, and so forth). Alteration of the parameter relative to a control cell or a reference parameter (e.g., a reference value) indicates that the test compound can modulate the responsiveness of the cell.

Still other cell-based assays including contacting cells with the test compound and evaluating resistance to a stress, for example, hypoxia, DNA damage (genotoxic stress), or oxidative stress. For example, it is possible to determine whether hypoxia-mediated cell death is attenuated by the test compound.

Cell-Free Assays. In addition to cell-based assays, cell-free assays can also be used. In one example, the ability of the test compound to modulate interaction between a first GH/IGF-1 axis component and a second axis component is evaluated, e.g., interaction between GH and the GH receptor or GHRH and the GHRH receptor. This type of assay can be accomplished, for example, by coupling one of the components, with a radioisotope or enzymatic label such that binding of the labeled component to the other GH/IGF-1 axis component can be determined by detecting the labeled compound in a complex. A GH/IGF-1 axis component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product.

Competition assays can also be used to evaluate a physical interaction between a test compound and a target. For example, Pong et al. (1996) Mol Endocrinol 10:57 describes an assay which detects the displacement of a radiolabeled MK-0677 molecule from pituitary membranes.

In yet another embodiment, a cell-free assay is provided in which a GH/IGF-1 axis protein or biologically active portion thereof is contacted with a test compound and the ability of the test compound to bind to the GH/IGF-1 axis protein or biologically active portion thereof is evaluated. Exemplary biologically active portions of the GH/IGF-1 axis proteins to be used in assays include fragments which participate in interactions with non-GH/IGF-1 axis molecules, e.g., an ectodomain of a cell surface receptor, a cytoplasmic domain of a cell surface receptor, a kinase domain, and so forth.

Soluble and/or membrane-bound forms of isolated proteins (e.g., GH/IGF-1 axis components and their receptors or biologically active portions thereof) can be used in the cell-free assays. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate. In another example, the axis component can reside in a membrane, e.g., a liposome or other vesicle.

Cell-free assays involve preparing a reaction mixture of the target protein (e.g., the GH/IGF-1 axis component) and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999)Comb Chem HTS 2:177-190; Jameson et al. (1995) Methods Enzymol 246:283; Seethala et al. (1998) Anal Biochem. 255:257. Fluorescence polarization can be monitored in multiwell plates, e.g., using the Tecan Polarion™ reader. See, e.g., Parker et al. (2000) Journal of Biomolecular Screening 5:77-88; and Shoeman, et al. (1999) 38, 16802-16809.

In another embodiment, determining the ability of the GH/IGF-1 axis component protein to bind to a target molecule can be accomplished using real-time Biomolecular Interaction Analysis (BIA) (see, e.g., Sjolander, S, and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the axis component is anchored onto a solid phase. The axis component/test compound complexes anchored on the solid phase can be detected at the end of the reaction, e.g., the binding reaction. For example, the axis component can be anchored onto a solid surface, and the test compound, (which is not anchored), can be labeled, either directly or indirectly, with detectable labels discussed herein.

It may be desirable to immobilize either the GH/IGF-1 axis component or an anti-GH/IGF-1 axis component antibody to facilitate separation of complexed from uncomplexed forms of one or both of the proteins, as well as to accommodate automation of the assay. Binding of a test compound to a GH/IGF-1 axis component protein, or interaction of a GH/IGF-1 axis component protein with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/GH/IGF-1 axis component fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or GH/IGF-1 axis component protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of GH/IGF-1 axis component binding or activity determined using standard techniques.

Other techniques for immobilizing either a GH/IGF-1 axis component protein or a target molecule on matrices include using conjugation of biotin and streptavidin. Biotinylated GH/IGF-1 axis component protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a GH/IGF-1 axis component protein or target molecules but which do not interfere with binding of the GH/IGF-1 axis component protein to its target molecule. Such antibodies can be derivatized to the wells of the plate, and unbound target or the GH/IGF-1 axis component protein trapped in the wells by antibody conjugation. Methods for detecting such complexes, in addition to those described above for the GST-immobilized complexes, include immunodetection of complexes using antibodies reactive with the GH/IGF-1 axis component protein or target molecule, as well as enzyme-linked assays which rely on detecting an enzymatic activity associated with the GH/IGF-1 axis component protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the GH/IGF-1 axis component protein or biologically active portion thereof with a known compound which binds a GH/IGF-1 axis component to form an assay mixture, contacting the assay mixture with a test compound, and determining the ability of the test compound to interact with a GH/IGF-1 axis component protein, wherein determining the ability of the test compound to interact with the GH/IGF-1 axis component protein includes determining the ability of the test compound to preferentially bind to the GH/IGF-1 axis component or biologically active portion thereof, or to modulate the activity of a target molecule, as compared to the known compound.

The target products can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target product. Such compounds can include, but are not limited to molecules such as antibodies, peptides, and small molecules. The preferred targets/products for use in this embodiment are the GH/IGF-1 axis components. Also disclosed are methods for determining the ability of the test compound to modulate the activity of a GH/IGF-1 axis component protein through modulation of the activity of a downstream effector of a GH/IGF-1 axis component target molecule. For example, the activity of the effector molecule on an appropriate target can be determined, or the binding of the effector to an appropriate target can be determined, as previously described.

To identify compounds that interfere with the interaction between the target product and its cellular or extracellular binding partner(s), a reaction mixture containing the target product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target product can also be compared to complex formation within reaction mixtures containing the test compound and mutant target product. This comparison can be important in those cases wherein it is desirable to identify compounds that disrupt interactions of mutant but not normal target products.

These assays can be conducted in a heterogeneous or homogeneous format. Heterogeneous assays involve anchoring either the target product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In another embodiment, a homogeneous assay can be used. For example, a preformed complex of the target product and the interactive cellular or extracellular binding partner product is prepared in that either the target products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target product-binding partner interaction can be identified.

In yet another aspect, the GH/IGF-1 axis component proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with the GH/IGF-1 axis component (“GH/IGF-1 axis component-binding proteins” or “GH/IGF-1 axis component-bp”) and are involved in GH/IGF-1 axis component activity. Such GH/IGF-1 axis component-bps can be activators or inhibitors of signals by the GH/IGF-1 axis component proteins or GH/IGF-1 axis component targets as, for example, downstream elements of a GH/IGF-1 axis component-mediated signaling pathway.

The two-hybrid system is based on the modular nature of most transcription factors, which consist of separable DNA-binding and activation domains. Briefly, the assay utilizes two different DNA constructs. In one construct, the gene that codes for a GH/IGF-1 axis component protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: GH/IGF-1 axis component protein can be the fused to the activator domain.) If the “bait” and the “prey” proteins are able to interact, in vivo, forming a GH/IGF-1 axis component-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the GH/IGF-1 axis component protein. In another embodiment, the two-hybrid assay is used to monitor an interaction between two components of the axis that are known to interact. The two hybrid assay is conducted in the presence of a test compound, and the assay is used to determine whether the test compound enhances or diminishes the interaction between the components.

In another embodiment, modulators of a GH/IGF-I axis component gene expression are identified. For example, a cell or cell free mixture is contacted with a candidate compound and the expression of the GH/IGF-I axis component mRNA or protein evaluated relative to the level of expression of GH/IGF-I axis component mRNA or protein in the absence of the candidate compound. When expression of the GH/IGF-I axis component mRNA or protein is greater in the presence of the candidate compound than in its absence, the candidate compound is identified as a stimulator of GH/IGF-I axis component mRNA or protein expression. Alternatively, when expression of the GH/IGF-I axis component mRNA or protein is less (statistically significantly less) in the presence of the candidate compound than in its absence, the candidate compound is identified as an inhibitor of the GH/IGF-I axis component mRNA or protein expression. The level of the GH/IGF-I axis component mRNA or protein expression can be determined by methods for detecting GH/IGF-I axis component mRNA or protein.

Organismal Assays. Still other methods for evaluating a test compound include organismal based assays, e.g., using a mammal (e.g., a mouse, rat, primate, or other non-human), or other animal (e.g., Xenopus, zebrafish, or an invertebrate such as a fly or nematode). In some cases, the organism is a transgenic organism, e.g., an organism which includes a heterologous GH/IGF-1 axis component, (e.g., from a mammal, e.g., a human). The test compound can be administered to the organism once or as a regimen (regular or irregular). A parameter of the organism is then evaluated, e.g., an age-associated parameter, a parameter of the GH/IGF-1 axis, a parameter related to neurological function or to polyglutamine aggregation. Test compounds that are indicated as of interest result in a change in the parameter relative to a reference, e.g., a parameter of a control organism. Other parameters (e.g., related to toxicity, clearance, and pharmacokinetics) can also be evaluated.

The organism can also include a reporter protein that includes a polyglutamine repeat region which has at least 35 glutamine (e.g., as described below), e.g., at least 40, 50, 60, 65, 70 or 80 glutamines.

In some embodiment, the test compound is evaluated using an animal that has a particular disorder, e.g., a disorder mediated by polyglutamine aggregation or a neurodegenerative disorder. These disorders provide a sensitized system in which the test compound's effects on physiology can be observed. Exemplary disorders include: denervation, disuse atrophy; metabolic disorders (e.g., disorder of obese and/or diabetic animals such as db/db mouse and ob/ob mouse); cerebral, liver ischemia; cisplatin/taxol/vincristine models; various tissue (xenograph) transplants; transgenic bone models; Pain syndromes (include inflammatory and neuropathic disorders); Paraquat, genotoxic, oxidative stress models; pulmonary obstruction (e.g., asthma models); and polyglutamine aggregation models (see, e.g., below).

In one embodiment, the parameter is associated with polyglutamine aggregation of a neurodegenerative disorder. A test compound that is favorably indicated can cause an amelioration of the symptom relative to a similar reference animal not treated with the compound. In a related embodiment, the parameter is a parameter of the GH/IGF-1 axis or an age-associated parameter. Exemplary parameters associated with the function of GH/IGF-1 axis include GH concentration, IGF-1 concentration, GHSH concentration, and so forth.

In assessing whether a test compound is capable of inhibiting the GH/IGF-1 axis for the purpose of modulating polyglutamine aggregation, a number of age-associated parameters or biomarkers can be monitored or evaluated. Exemplary age associated parameters include: (i) lifespan of the cell or the organism; (ii) presence or abundance of a gene transcript or gene product in the cell or organism that has a biological age-dependent expression pattern; (iii) resistance of the cell or organism to stress; (iv) one or more metabolic parameters of the cell or organism; (v) proliferative capacity of the cell or a set of cells present in the organism; and (vi) physical appearance or behavior of the cell or organism. Similarly it is possible evaluate biomarkers that are correlated with polyglutamine aggregation or neurodegenerative disorders.

The term “average lifespan” refers to the average of the age of death of a cohort of organisms. In some cases, the “average lifespan” is assessed using a cohort of genetically identical organisms under controlled environmental conditions. Deaths due to mishap are discarded. For example, with respect to a nematode population, hermaphrodites that die as a result of the “bag of worms” phenotype are typically discarded. Where average lifespan cannot be determined (e.g., for humans) under controlled environmental conditions, reliable statistical information (e.g., from actuarial tables) for a sufficiently large population can be used as the average lifespan.

Characterization of molecular differences between two such organisms, e.g., one reference organism and one organism treated with a GH/IGF-1 axis modulator can reveal a difference in the physiological state of the organisms. The reference organism and the treated organism are typically the same chronological age. The term “chronological age” as used herein refers to time elapsed since a preselected event, such as conception, a defined embryological or fetal stage, or, more preferably, birth. A variety of criteria can be used to determine whether organisms are of the “same” chronological age for the comparative analysis. Typically, the degree of accuracy required is a function of the average lifespan of a wild-type organism. For example, for the nematode C. elegans, for which the laboratory wild-type strain N2 lives an average of about 16 days under some controlled conditions, organisms of the same age may have lived for the same number of days. For mice, organism of the same age may have lived for the same number of weeks or months; for primates or humans, the same number of years (or within 2, 3, or 5 years); and so forth. Generally, organisms of the same chronological age may have lived for an amount of time within 15, 10, 5, 3, 2 or 1% of the average lifespan of a wild-type organism of that species. In a preferred embodiment, the organisms are adult organisms, e.g. the organisms have lived for at least an amount of time in which the average wild-type organism has matured to an age at which it is competent to reproduce.

In some embodiments, the organismal screening assay is performed before the organisms exhibit overt physical features of aging. For example, the organisms may be adults that have lived only 10, 30, 40, 50, 60, or 70% of the average lifespan of a wild-type organism of the same species.

Age-associated changes in metabolism, immune competence, and chromosomal structure have been reported. Any of these changes can be evaluated, either in a test subject (e.g., for an organism based assay), or for a patient (e.g., prior, during and/or after treatment with a therapeutic described herein).

In another embodiment, a marker associated with caloric restriction is evaluated in a subject organism of a screening assay (or a treated subject). Although these markers may not be age-associated, they may be indicative of a physiological state that is altered when the GH/IGF-1 axis is modulated. The marker can be an mRNA or protein whose abundance changes in calorically restricted animals. WO 01/12851 and U.S. Pat. No. 6,406,853 describe exemplary markers.

Differences in aging (e.g., age-associated parameters and biomarkers) can indicate that cells have altered ability to regulate polyglutamine aggregation.

Evaluating Polyglutamine Aggregation

A variety of cell free assays, cell based assays, and organismal assays are available for evaluating polyglutamine aggregation, e.g., Huntingtin polyglutamine aggregation. Some examples are described, e.g., in U.S. 2003-0109476.

Assays (e.g., cell free, cell-based, or organismal) can include a reporter protein that includes a polyglutamine repeat region which has at least 35 polyglutamines. The reporter protein can be easily detectable, e.g., by fluorescence. For example, the protein is conjugated to a fluorophore, for example, fluorescein isothiocyanate (FITC), allophycocyanin (APC), R-phycoerythrin (PE), peridinin chlorophyll protein (PerCP), Texas Red, Cy3, Cy5, Cy7, or a fluorescence resonance energy tandem fluorophore such as PerCP-Cy5.5, PE-Cy5, PE-Cy5.5, PE-Cy7, PE-Texas Red, and APC-Cy7. In another example the protein is “intrinsically fluorescent” in that it has a chromophore is entirely encoded by its amino acid sequence and can fluoresce without requirement for cofactor or substrate. For example, the protein can include a green fluorescent protein (GFP)-like chromophore. As used herein, “GFP-like chromophore” means an intrinsically fluorescent protein moiety comprising an 11-stranded β-barrel with a central α-helix, the central α-helix having a conjugated π-resonance system that includes two aromatic ring systems and the bridge between them.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation. The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999). A variety of such modified chromophores are now commercially available and can readily be used in the fusion proteins.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins that further include a polyglutamine region. A variety of EGFP vectors, both plasmid and viral, are available commercially (Clontech Labs, Palo Alto, Calif., USA). Still other engineered GFP proteins are known. See, e.g., Heim et al., Curr. Biol. 6:178-182 (1996); Cormack et al., Gene 173:33-38 (1996), BFP2, EYFP (“enhanced yellow fluorescent protein”), EBFP, Ormo et al., Science 273:1392-1395 (1996), Heikal et al., Proc. Natl. Acad. Sci. USA 97:11996-12001 (2000). ECFP (“enhanced cyan fluorescent protein”) (Clontech Labs, Palo Alto, Calif., USA). The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048.

In one embodiment, a reporter protein that includes a polyglutamine repeat region which has at least 35 polyglutamines is used in a cell-based assay.

In one example, PC12 neuronal cell lines that have a construct engineered to express a protein encoded by HD gene exon 1 containing alternating, repeating codons (e.g., repeats of “CAA CAG CAG CAA CAG CAA”, SEQ ID NO:2) fused to an enhanced GFP (green fluorescent protein) gene can be used. See, e.g., Boado et al. J. Pharmacol. and Experimental Therapeutics 295(1): 239-243 (2000) and Kazantsev et al. Proc. Natl. Acad. Sci. USA 96: 11404-09 (1999). Expression of this gene leads to the appearance of green fluorescence co-localized to the site of protein aggregates. The HD gene exon 1-GFP fusion gene is under the control of an inducible promoter regulated by muristerone. A particular construct has approximately 46 glutamine repeats (encoded by either CAA or CAG). Other constructs have, for example, 103 glutamine repeats. PC12 cells are grown in DMEM, 5% Horse serum (heat inactivated), 2.5% FBS and 1% Pen-Strep, and maintained in low amounts on Zeocin and G418. The cells are plated in 24-well plates coated with poly-L-lysine coverslips, at a density of 5·10⁵ cells/ml in media without any selection. Muristerone is added after the overnight incubation to induce the expression of HD gene exon 1-GFP. The cells can be contacted with a test compound, e.g., before or after plating and before or after induction. The data can be acquired on a Zeiss inverted 100M Axioskop equipped with a Zeiss 510 LSM confocal microscope and a Coherent Krypton Argon laser and a Helium Neon laser. Samples can be loaded into Lab-Tek II chambered coverglass system for improved imaging. The number of Huntingtin-GFP aggregations within the field of view of the objective is counted in independent experiments (e.g., at least three or seven independent experiments).

Other exemplary means for evaluating samples include a high throughput apparatus, such as the Amersham Biosciences IN-CELL™ Analysis System and CELLOMICS™ ArrayScan HCS System which permit the subcellular location and concentration of fluorescently tagged moieties to be detected and quantified, both statically and kinetically. See also, U.S. Pat. No. 5,989,835.

Other exemplary mammalian cell lines include: a CHO cell line and a 293 cell line. For example, CHO cells with integrated copies of HD gene exon 1 with approximately 103Q repeats fused to GFP as a fusion construct encoding HD gene exon 1 Q103-GFP produce a visible GFP aggregation at the nuclear membrane, detectable by microscopy, whereas CHO cells with integrated copies of fusion constructs encoding HD gene exon 1 Q24-GFP in CHO cells do not produce a visible GFP aggregation at the nuclear membrane. In another example, 293 cells with integrated copies of the HD gene exon 1 containing 84 CAG repeats are used.

A number of animal model system for Huntington's disease are available. See, e.g., Brouillet, Functional Neurology 15(4): 239-251 (2000); Ona et al. Nature 399: 263-267 (1999), Bates et al. Hum Mol Genet. 6(10):1633-7 (1997); Hansson et al. J. of Neurochemistry 78: 694-703; and Rubinsztein, D. C., Trends in Genetics, Vol. 18, No. 4, pp. 202-209 (a review on various animal and non-human models of HD).

In one embodiment, the animal is a transgenic mouse that can express (in at least one cell) a human Huntingtin protein, a portion thereof, or fusion protein comprising human Huntingtin protein, or a portion thereof, with, for example, at least 36 glutamines (e.g., encoded by CAG repeats (alternatively, any number of the CAG repeats may be CAA) in the CAG repeat segment of exon 1 encoding the polyglutamine tract).

An example of such a transgenic mouse strain is the R6/2 line (Mangiarini et al. Cell 87: 493-506 (1996)). The R6/2 mice are transgenic Huntington's disease mice, which over-express exon one of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease. Abnormal aggregates, constituted in part by the N-terminal part of Huntingtin (encoded by HD exon 1), are observed in R6/2 mice, both in the cytoplasm and nuclei of cells (Davies et al. Cell 90: 537-548 (1997)). For example, the human Huntingtin protein in the transgenic animal is encoded by a gene that includes at least 55 CAG repeats and more preferably about 150 CAG repeats.

These transgenic animals can develop a Huntington's disease-like phenotype. These transgenic mice are characterized by reduced weight gain, reduced lifespan and motor impairment characterized by abnormal gait, resting tremor, hindlimb clasping and hyperactivity from 8 to 10 weeks after birth (for example the R6/2 strain; see Mangiarini et al. Cell 87: 493-506 (1996)). The phenotype worsens progressively toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. Accordingly, evaluating can include assessing parameters related to neurotransmitter levels, neurotransmitter receptor levels, brain size and striatum size. In addition, abnormal aggregates containing the transgenic part of or full-length human Huntingtin protein are present in the brain tissue of these animals (e.g., the R6/2 transgenic mouse strain). See, e.g., Mangiarini et al. Cell 87: 493-506 (1996), Davies et al. Cell 90: 537-548 (1997), Brouillet, Functional Neurology 15(4): 239-251 (2000) and Cha et al. Proc. Natl. Acad. Sci. USA 95: 6480-6485 (1998).

To test the effect of the test compound or known compound described in the application in an animal model, different concentrations of test compound are administered to the transgenic animal, for example by injecting the test compound into circulation of the animal. In one embodiment, a Huntington's disease-like symptom is evaluated in the animal. For example, the progression of the Huntington's disease-like symptoms, e.g. as described above for the mouse model, is then monitored to determine whether treatment with the test compound results in reduction or delay of symptoms. In another embodiment, disaggregation of the Huntingtin protein aggregates in these animals is monitored. The animal can then be sacrificed and brain slices are obtained. The brain slices are then analyzed for the presence of aggregates containing the transgenic human Huntingtin protein, a portion thereof, or a fusion protein comprising human Huntingtin protein, or a portion thereof. This analysis can includes, for example, staining the slices of brain tissue with anti-Huntingtin antibody and adding a secondary antibody conjugated with FITC which recognizes the anti-Huntingtin's antibody (for example, the anti-Huntingtin antibody is mouse anti-human antibody and the secondary antibody is specific for human antibody) and visualizing the protein aggregates by fluorescent microscopy. Alternatively, the anti-Huntingtin antibody can be directly conjugated with FITC. The levels of Huntingtin's protein aggregates are then visualized by fluorescent microscopy.

A Drosophila melanogaster model system for Huntington's disease is also available. See, e.g., Steffan et al., Nature, 413: 739-743 (2001) and Marsh et al., Human Molecular Genetics 9: 13-25 (2000). For example, a transgenic Drosophila can be engineered to express human Huntingtin protein, a portion thereof (such as exon 1), or fusion protein comprising human Huntingtin protein, or a portion thereof, with, for example, a polyglutamine region that includes at least 36 glutamines (e.g., encoded by CAG repeats (preferably 51 repeats or more) (alternatively, any number of the CAG repeats may be CAA)) The polyglutamine region can be encoded by the CAG repeat segment of exon 1 encoding the poly Q tract. These transgenic flies can also engineered to express human Huntingtin protein, a portion thereof (such as exon 1), or fusion protein comprising human Huntingtin protein, or a portion thereof, in neurons, e.g., in the Drosophila eye.

The test compound (e.g., different concentrations of the test compound) or a compound described herein can be administered to the transgenic Drosophila, for example, by applying the pharmaceutical compositions that include the compound into to the animal or feeding the compound as part of food. Administration of the compound can occur at various stages of the Drosophila life cycle. The animal can be monitored to determine whether treatment with the compound results in reduction or delay of Huntington's disease-like symptoms, disaggregation of the Huntingtin protein aggregates, or reduced lethality and/or degeneration of photoreceptor neurons are monitored.

Neurodegeneration due to expression of human Huntingtin protein, a portion thereof (such as exon 1), or fusion protein comprising human Huntingtin protein, or a portion thereof, is readily observed in the fly compound eye, which is composed of a regular trapezoidal arrangement of seven visible rhabdomeres (subcellular light-gathering structures) produced by the photoreceptor neurons of each Drosophila ommatidium. Expression of human Huntingtin protein, a portion thereof (such as exon 1), or fusion protein comprising human Huntingtin protein, or a portion thereof, leads to a progressive loss of rhabdomeres. Thus, an animal to which a test compound is administered can be evaluated for neuronal degeneration.

Antibodies

Immunoglobulins can also be produced that bind to a GH/IGF-1 axis component and, for example, reduce axis activity. For example, an immunoglobulin can bind to a GH receptor and prevent GH binding, without itself activating the receptor. Similarly, an immunoglobulin can bind to a secreted axis component, e.g., GH itself and so forth. In other examples, the immunoglobulin can function be recruit an effector activity, e.g., complement or a cytotoxic lymphocyte. In a preferred embodiment, the immunoglobulin is human or non-antigenic in the subject.

An immunoglobulin can be, for example, an antibody or an antigen-binding fragment thereof. As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids). The term “antigen-binding fragment” of an antibody (or simply “antibody portion,” or “fragment”), as used herein, refers to one or more fragments of a full-length antibody that retain the ability to specifically bind to the antigen. Examples of antigen-binding fragments include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

In one embodiment, the antibody against the axis component is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey). Preferably, the non-human antibody is a rodent (mouse or rat antibody). Method of producing rodent antibodies are known in the art. Human monoclonal antibodies can be generated using transgenic mice carrying the human immunoglobulin genes rather than the mouse system (see, e.g., WO 91/00906 and WO 92/03918). Other methods for generating immunoglobulin ligands include phage display (e.g., as described in U.S. Pat. No. 5,223,409 and WO 92/20791).

RNAi

It is also possible to attenuate GH/IGF-1 axis activity using a double-stranded RNA (dsRNA) that mediates RNA interference (RNAi). The dsRNA can be delivered to cells or to an organism. Endogenous components of the cell or organism can trigger RNA interference (RNAi) which silences expression of genes that include the target sequence. dsRNA can be produced by transcribing a cassette in both directions, for example, by including a T7 promoter on either side of the cassette. The insert in the cassette is selected so that it includes a sequence from a GH/IGF-1 axis component to be attenuated. The sequence need not be full length, for example, an exon, or at least 50 nucleotides, preferably from the 5′ half of the transcript, e.g., within 300 nucleotides of the ATG. See also, the HiScribe™ RNAi Transcription Kit (New England Biolabs, MA) and Fire, A. (1999) Trends Genet. 15, 358-363. dsRNA can be digested into smaller fragments. See, e.g., US Patent Application 2002-0086356 and 2003-0084471. In one embodiment, an siRNA is used. siRNAs are small double stranded RNAs (dsRNAs) that optionally include overhangs. For example, the duplex region is about 18 to 25 nucleotides in length, e.g., about 19, 20, 21, 22, 23, or 24 nucleotides in length. Typically the siRNA sequences are exactly complementary to the target mRNA.

dsRNAs can be used to silence gene expression in mammalian cells. See, e.g., Clemens, J. C. et al. (2000) Proc. Natl. Sci. USA 97, 6499-6503; Billy, E. et al. (2001) Proc. Natl. Sci. USA 98, 14428-14433; Elbashir et al. (2001) Nature. 411(6836):494-8; Yang, D. et al. (2002) Proc. Natl. Acad. Sci. USA 99, 9942-9947.

For example, double stranded RNA molecules complementary to a nucleic acid encoding GHRH, GHRH-R, GHS-R, GH, GH-R, IGF-1, IGF-1-R, PI(3) kinase, PDK1, or AKT-1,2,3 can be used to attenuate activity of the GH/IGF-1 axis.

Stem Cell Therapy

It is also possible to modify stem cells using nucleic acid recombination, e.g., to insert a transgene. The modified stem cell can be administered to a subject. Methods for cultivating stem cells in vitro are described, e.g., in US Application 2002-0081724. In some examples, the stem cells can be induced to differentiate in the subject and express the transgene.

Pharmaceutical Compositions

A compound that modulates the GH/IGF-1 axis can be incorporated into a pharmaceutical composition for administration to a subject, e.g., a human, a non-human animal, e.g., an animal patient (e.g., pet or agricultural animal) or an animal model (e.g., an animal model for polyglutamine aggregation disorder or a neurodegenerative disorder. Such compositions typically include a small molecule (e.g., a small molecule that is a GH/IGF-1 antagonist), nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Exemplary compounds that can be used for treatment include: Pegvisomant, a somatostatin agonist (such as L-054,522), an IGF-1R competitive inhibitor such as Tyrphostin AG 538 (see, e.g., Biochemistry 2000.39.15705) or Tyrphostin AG 1024 (Br J Cancer 2001 Dec. 14; 85(12):2017-21) an IGF-1R antagonist such as H-1356 (see, e.g., Diabetes Res Clin Pract 2002 February; 55(2):89-98) and hetero-aryl-aryl ureas (see, e.g., U.S. Pat. No. 6,337,338), a Akt modulator such as trisenox (see, e.g., Blood 2001, 98:618) or UCN-01 (e.g., mediating dephosphorylation and inactivation of AKT; Oncogene 2002.21.1727), or a PI(3) kinase inhibitor, e.g., LY294002 (Mol Endocrinol 2002 February; 16(2):342-52) or Wortmannin (see, e.g., J Cell Biochem 2002; 84:708-16), a GHRH antagonist peptide such as JV-1-36, JV-1-38 (Proc. Natl. Acad. Sci. USA 1999 96:692); a GHRH/GHRH receptor antagonist such as GHRH-44 (see, e.g., J Clin Endocrinol Metab 2001 November; 86(11):5485-90); an inhibitor of GH release such as CST-14 (cortistatin-14); Sandostatin LAR; a somatostatin-analogist cyclic peptide e.g., as described in U.S. Pat. No. 5,962,409; octreotide acetate; slow release analog of somatostatin such as SR-lancreotide, BIM 23014 or another compound, e.g., a compound described herein.

TABLE 2
Exemplary Compounds

| Description | Compound | Source |
|---|---|---|
| Somatostatin-analogous cyclic | | Zentaris |
| peptides with inhibitory activity on | | |
| GH | | |
| IGF-1 receptor antagonist | H-1356 cyclic peptide, C- | Bachem Bioscience |
| | T-A-A-P-L-K-P-A-K-S- | |
| | C- (SEQ ID NO: 3) | |
| Inhibitor of IGF-1R | Tyrphostin AG 1024 | Alexis Biochemicals, Calbiochem |
| GHRH receptor antagonist | GHRH antagonist and | GHRH antagonist from Bachem |
| | GHRH-44 | Bioscience; GHRH-44 from Peninsula |
| | | Laboratories |
| GH receptor antagonist | pegvisomant | Pharmacia |
| IGF-1R antagonists | heteroaryl-aryl ureas | Telik, Inc. |
| Janus-kinase-3 inhibitor | WHI-P154 | Calbiochem #420104 |
| dephosphorylation and inactivation | UCN-01 -- 7- | Kyowa Hakko |
| of Akt | hydroxystaurosporine | |
| IGF-1R competitive inhibitor | tyrphostin AG 538 | Calbiochem AG538 Cat #658403, I- |
| | | OMe 538 Cat # 658417 |
| Inhibitor of GH release in rats | CST-14 (cortistatin-14) | Penlabs; CAT. No. 8027 |
| Sandostatin LAR | octreotide acetate | Novartis; Penlabs - CAT. No. 8060 |
| AKT inhibitor | trisenox | Marketer - Cell Therapeutics |
| Modulator of GH release | Somatostatin | Somatostatins from Peninsula Labs |
| | | (Penlabs) |
| slow release analog of somatostatin | SR-lancreotide, BIM | Beaufour Ipsen |
| | 23014 | |
| GHRH antagonist peptides | JV-1-36, JV-1-38 | Phoenix peptide |

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells, e.g., a pituitary cell) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7.sup.th ed., Lippincott Williams & Wilkins Publishers (1999) (ISBN: 0683305727); and Kibbe (ed.), Handbook of Pharmaceutical Excipients American Pharmaceutical Association, 3.sup.rd ed. (2000) (ISBN: 091733096X).

Toxicity and therapeutic efficacy of compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in a therapeutic method, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments.

For antibody compounds that modulate the axis, one preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

Agents that modulate expression or activity can be used. An agent may, for example, be a small molecule. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules that modulate the GH/IGF-1 axis can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

A modulator of the GH/IGF-1 axis that reduces polyglutamine aggregation, e.g., a modulator described herein, can be provided in a kit. The kit includes: (a) the modulator, e.g., a composition that includes the modulator, and (b) informational material. The informational material can be descriptive, instructional, marketing or other material that relates to the methods described herein and/or the use of the modulator for the methods described herein. For example, the informational material describes methods for administering the modulator to reduce polyglutamine aggregation or to treat or prevent a neurodegenerative disorder, e.g., Huntington's disease.

In one embodiment, the informational material can include instructions to administer the modulator in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., a human having, or at risk for a neurodegenerative disorder or a polyglutamine aggregation-based disorder. For example, the human is an adult, e.g., an adult with normal GH/IGF-1 axis activity for the adult's age, or with abnormal axis activity (e.g., above average activity for the adult's age). The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the modulator, the composition of the kit can include other ingredients, such as a solvent or buffer, a stabilizer or a preservative, and/or a second agent for treating a condition or disorder described herein, e.g. neurodegenerative disorder or a polyglutamine aggregation-based disorder. Alternatively, the other ingredients can be included in the kit, but in different compositions or containers than the modulator. In such embodiments, the kit can include instructions for admixing the modulator and the other ingredients, or for using the modulator together with the other ingredients.

The modulator can be provided in any form, e.g., liquid, dried or lyophilized form. It is preferred that the modulator be substantially pure and/or sterile. When the modulator is provided in a liquid solution, the liquid solution preferably is an aqueous solution, with a sterile aqueous solution being preferred. When the modulator is provided as a dried form, reconstitution generally is by the addition of a suitable solvent. The solvent, e.g., sterile water or buffer, can optionally be provided in the kit.

The kit can include one or more containers for the composition containing the modulator. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the modulator. For example, the kit includes a plurality of syringes, ampules, foil packets, or blister packs, each containing a single unit dose of the modulator. The containers of the kits can be air tight and/or waterproof.

The compositions can be administered to a subject, e.g., an adult subject, particularly a subject having or at risk for having a neurodegenerative disorder or a polyglutamine aggregation-based disorder. The method can include evaluating a subject (or genetic relative of the subject), e.g., to characterize a symptom of the neurodegenerative disorder or polyglutamine aggregation-based disorder, or other marker of the disorder (e.g., a genetic marker), and thereby identifying a subject as having the disorder or being at risk for the disorder. Exemplary neurodegenerative disorder or a polyglutamine aggregation-based disorder are described above.

Evaluating Huntington's Disease

A variety of methods are available to evaluate and/or monitor Huntington's disease. A variety of clinical symptoms and indicia for the disease are known. Huntington's disease causes a movement disorder, psychiatric difficulties and cognitive changes. The degree, age of onset, and manifestation of these symptoms can vary. The movement disorder can include quick, random, dance-like movements called chorea.

One method for evaluating Huntington's disease uses the Unified Huntington's disease Rating Scale (UNDRS). It is also possible to use individual tests alone or in combination to evaluate if at least one symptom of Huntington's disease is ameliorated. The UNDRS is described in Movement Disorders (vol. 11:136-142, 1996) and Marder et al. Neurology (54:452-458, 2000). The UNDRS quantifies the severity of Huntington's Disease. It is divided into multiple subsections: motor, cognitive, behavioral and functional. In one embodiment, a single subsection is used to evaluate a subject. These scores can be calculated by summing the various questions of each section. Some sections (such as chorea and dystonia) can include grading each extremity, face, bucco-oral-ligual, and trunk separately.

Exemplary motor evaluations include: ocular pursuit, saccade initiation, saccade velocity, dysarthria, tongue protrusion, finger tap ability, pronate/supinate, a fist-hand-palm sequence, rigidity of arms, bradykinesia, maximal dystonia (trunk, upper and lower extremities), maximal chorea (e.g., trunk, face, upper and lower extremities), gait, tandem walking, and retropulsion.

An exemplary treatment can cause a change in the Total Motor Score 4 (TMS-4), a subscale of the UHDRS, e.g., over a one-year period.

### EXAMPLES

The inventions are further described in the following examples, which do not limit the scope of the inventions described in the claims.

### Example 1

### In Vitro Determination of GH/IGF-1 Antagonist Activity

A commonly used method to screen for compounds that affect GH secretion is to use the rat pituitary cell culture assay. In a typical experiment involving rat pituitary cell culture assays to determine a compound's effect on GH secretion, pituitary glands are aseptically removed from Wistar male rats (150-200 g) and cultures of pituitary cells are prepared according to Cheng et al. Endocrinology 124: 2791-2798, 1989. The cells are treated with various compounds and assayed for GH secreting activity as described by Cheng et al., infra.

Functional activity of the various compounds can be evaluated by measuring GH secretion from primary cultures of rat anterior pituitary cells (Yang et al. Proc. Natl. Acad. Sci. USA 95:10836-10841, 1998). Cells are isolated from rat pituitaries by enzymatic digestion with 0.2% collagenase and 0.2% hyaluronidase in Hanks' balanced salt solution. The cells are suspended in culture medium and adjusted to a concentration of 1.5 3 105 cells/ml and 1.0 ml of this suspension is placed in each well of a 24-well tray. Cells are maintained in a humidified 5% CO2/95% air atmosphere at 37° C. for 3-4 days. The culture medium consists of DMEM containing 0.37% NaHCO3, 10% horse serum, 2.5% fetal bovine serum, 1% nonessential amino acids, 1% glutamine, 1% nystatin, and 0.1% gentamicin. Before testing compounds for their capacity to inhibit GH release, cells are washed twice 1.5 hr before and once more immediately before the start of the experiment with the above culture medium containing 25 mM HEPES (pH 7.4). Compounds are tested in quadruplicate by adding them in 1 ml of fresh medium to each well and incubating them at 37° C. for 2 hr followed by centrifugation at 2000×g for 15 min to remove any cellular material. The supernatant fluid is assayed for GH by a double antibody radioimmunoassay. For example, antibody to rat GH (anti-rat GH-RIA-5/AFP-411S, hormones for iodination (rat GH-I-6/AFP-5676B), and reference preparation (rat GH-RP-2/AFP-3190B) can be used for this assay.

Other means of determining effects of a compound on GH are to use cultured human fetal pituitary cells or cultured GH-adenoma cells collected from acromegalic patients. Isolation of cells and growth conditions may differ from that described above.

In another example, the superfused rat pituitary system can be used to evaluate antagonism of the GH/IGF-1 axis by a compound (Vigh and Schally, Peptides 5:241-247, 1984; Rekasi and Schally, Proc. Natl. Acad. Sci. USA 90:2146-2149, 1993). Briefly, anterior pituitary cells are dispersed as described above. The test compound is perfused through the rat pituitary cells for 9 minutes (3 mL) at various concentrations (10⁻⁷-10⁻⁹ M). After this 9 minute incubation, the cells are exposed to a mixture of the test compound and 10⁻⁹ M hGHRH¹⁻²⁹NH₂ for an additional 3 minutes. To check the duration of the antagonistic effect of the test compound, 10⁻⁹M hGHRH¹⁻²⁹NH₂ is applied 30 and 60 minutes later for 3 minutes. GH content of the 1 mL fractions collected can be determined by double-antibody RIA (materials supplied by the National Hormone and Pituitary Program, Baltimore, Md.). Net integral values of the GH responses can be evaluated with a computer program designed for this use (Csernus, et al., Neuroendocrine Research Methods, ed. Greenstein (Harwood, London), 1991). GH responses can be compared to and expressed as a percentage of the original GH response induced by 10⁻⁹ M hGHRH¹⁻²⁹NH₂. The potencies of the test compounds can be compared to that of the standard GHRH antagonist (vide supra).

### Example 2

### GH/IGF-1 Axis Regulation of Polyglutamine Aggregation

Despite intense study, the nature of the transition of polyQ proteins to a toxic form is not well understood. Protein aggregates are a pathological hallmark of polyQ-mediated diseases (13), and aggregate formation has been observed in numerous in vitro and in vivo systems with polyQ proteins, leading to the hypothesis that the molecular events that lead to polyQ aggregation mark the transition to the toxic form (14). However, several studies have described an imperfect or inverse correlation between aggregate formation and toxicity (15-18). Thus, whether aggregate formation is necessary in the transition leading to diminished cellular function remains a central, but unresolved, question.

To address the underlying principles of polyQ-mediated aggregation and cellular toxicity, we have used Caenorhabditis elegans expressing chimeric fusions of polyQ and the yellow fluorescent protein (polyQ-YFP). Whereas previous studies on polyQ-mediated toxicity in animal models have compared the effects of short (<30Q) or long (>60Q) repeats, in this study we have analyzed transgenic lines expressing nine repeat lengths ranging from Q0 to Q82 with an emphasis on polyQ lengths in the range of 30 to 40 glutamine residues (Q29, Q33, Q35, Q40, Q44). We reasoned that such an analysis would allow us to test directly the polyQ threshold hypothesis and would yield insights into the nature of the transition governing conversion of polyQ-containing proteins to toxic forms.

Methods

DNA Cloning. Plasmids for expression of Q19-YFP and Q82-YFP in C. elegans body wall muscle were described (19). Constructs for expression of the repeat lengths were generated by PCR amplification of the appropriate polyQ-YFP cassette in pEYFP-N1 (CLONTECH) by using oligonucleotides containing restriction sites for NheI and KpnI. PCR amplicons were digested and ligated into the NheI and KpnI sites of pP30.38 containing the promoter and enhancer elements from the unc-54 myosin heavy-chain locus (20). RNA interference (RNAi) constructs were created by reverse transcription-PCR amplification of cDNA corresponding to age-1 (21) or daf-16 (22), digestion with KpnI/XbaI or SacI/SalI, respectively, and ligation into appropriately digested L4440 (ref. 23). Successful construction of plasmids was confirmed by DNA sequencing.

C. elegans Methods. Nematodes were handled by using standard methods (24). For generation of transgenic animals, plasmid DNAs encoding polyQ-YFP in pPD30.38 were linearized with PvuII and mixed (at 1 μml) with PvuII-digested C. elegans genomic DNA (100 μg/ml). Mixtures were microinjected into the gonads of adult hermaphrodite N2 or age-1 (hx546) animals. Transgenic F1 progeny were selected on the basis of fluorescence in muscle cells. Individual fluorescent F2 animals were picked to establish transgenic lines. At least three independent lines for each transgene were isolated and analyzed with similar results. Synchronized populations were isolated by collecting embryos from gravid adults after treatment with alkaline hypochlorite (2:5, vol/vol, bleach/1 M NaOH) for 10 min (25) or by collecting embryos laid by adult animals in a 6-h period. RNAi experiments were performed by growing animals on Escherichia coli strain HT115(DE3) transformed with the indicated plasmid or empty vector L4440 essentially as described (23).

Fluorescence Recovery After Photobleaching (FRAP). Animals were mounted on a 2% agar pad on a glass slide, immobilized in 1 mM levamisole, and subjected to FRAP analysis using a Zeiss LSM 510 confocal microscope imaged through a 40×1.0 numerical aperture objective with the 488-nm line for excitation. Areas indicated by boxes were bleached for 10 s at 100% power, and recovery images were acquired at the indicated times by using 7% power. Scanning time was 3 s.

Motility Assays. Individual animals were picked to fresh spread plates and their tracks were recorded at different intervals by using a charge-coupled device camera and Leica dissection stereomicroscope (magnification, ×8). Digital images of the tracks were analyzed to determine, the average velocity of the animals. Pixels in the images were converted to distances by using a ruler calibration macro in the OPENLAB™ (Improvision, Lexington, Mass.) software program. The distance traveled by each animal was determined by tracing its tracks in the image. Each data point was the average of two independent tracings of the same tracks. Dividing this distance by the time interval gave the motility index for each animal. Statistical significance of the results was determined by a χ² test. For blinded Q40 motility assays, adult animals were randomly picked from populations and measured for motility without knowledge of the aggregation phenotype. Once all motility assays were completed, the same animals were viewed by using fluorescence microscopy, and the number of aggregates was counted (see below). Because the polyQ transgenes were carried on extrachromosomal arrays, some animals in the population were nontransgenic and consequently provided internal controls. Motility values for wild-type (N2) and nontransgenic control groups were indistinguishable from one another.

Aggregate Quantitation. Animals were viewed at ×100 magnification with a stereomicroscope equipped for epifluorescence, and the number of polyQ aggregates was counted. Aggregates were defined as discrete structures with boundaries distinguishable from surrounding fluorescence on all sides. Aggregate size, measured by using confocal microscopy, typically ranged from 1 to 5 μm. At ×100 magnification, we were able to detect >80% of aggregates observable at higher magnifications. Repeated aggregate counts by the same observer and independent observers varied by less than 10%.

Results

Length-Dependent Threshold for Aggregation and Toxicity of polyQ Proteins. We previously described the formation of discrete cytoplasmic aggregates in body-wall muscle cells of C. elegans expressing Q82-YFP under the control of the unc-54 myosin heavy-chain promoter (19). We examined animals expressing Q0, Q19, Q29, Q33, Q35, Q40, Q44, Q64, and Q82 as chimeric fusions to YFP. In young adult animals (days 3-4) expressing repeats of Q35 or fewer, we observed diffuse fluorescence distribution in all expressing cells. In contrast, animals expressing Q44, Q64, or Q82 exhibited focal fluorescence distribution corresponding to protein aggregates. Q40 animals displayed a striking polymorphic distribution with diffuse fluorescence in some cells and foci in others. These results demonstrate a shift in the cellular distribution of the protein in young adult animals between Q35 and Q40.

The change from diffuse to focal fluorescence in animals expressing Q19 or Q82, respectively, corresponds to a conversion of the biochemical state of the polyQ proteins from soluble to aggregate as detected in whole animal extracts (19). However, to investigate whether the cell-to-cell variation observed in Q40 animals reflected different in vivo states of polyQ proteins, we used a noninvasive method, FRAP. We reasoned that soluble YFP-tagged proteins in the cytoplasm would diffuse freely and recover rapidly after photobleaching as has been demonstrated for green fluorescent protein in solution and in tissue culture cells (26). After photobleaching, the fluorescence of Q0-YFP and Q29-YFP recovered completely within 3 s, suggesting that both YFP alone (Q0) and Q29-YFP exhibited biochemical properties as soluble proteins in vivo. In contrast, fluorescence of Q82-YFP did not recover within 30 s after photobleaching and remained bleached after 5 min, consistent with its properties as an aggregate. These data indicated that FRAP could be used as a tool to distinguish between different states of Q40-YFP expressed in adjacent cells of individual animals. Whereas diffuse Q40-YFP exhibited rapid recovery after photobleaching, similar to that observed for Q0 and Q29, focal Q40-YFP exhibited slow recovery indicative of protein aggregates. Although FRAP does not directly assess biochemical properties, the different recovery rates observed for diffuse and focal Q40-YFP are consistent with different biochemical states of Q40 within adjacent cells of the same animal.

To determine whether the appearance of polyQ aggregates was associated with cellular dysfunction, we examined the motility of 4-day-old adult animals expressing polyQ tracts of 0, 19, 29, 35, 40, or 82 residues. C. elegans are maintained on agar plates with a lawn of E. coli. Consequently, as the animals move, their tracks can be visualized and quantified to establish a motility index. After 2 min, wild-type (N2) animals had moved 10 to 20 body lengths from the point of origin, whereas Q82 animals remained at or near the point of origin. Quantitation of these results revealed a ≈10-fold reduction in motility of young adult Q82 animals, corresponding to a defect similar to animals expressing mutant unc-54 myosin heavy chain. In contrast, Q19-, Q29-, or Q35-expressing animals that did not have polyQ aggregates exhibited motility similar to wild type. Q40 animals, which had aggregates in some cells but not in others, exhibited an intermediate motility defect with a high degree of variation in the intensity of loss of motility across a population.

Variation in Aggregate Formation Underlies Polymorphism in Q40-Mediated Motility Defect. In addition to cell-to-cell differences in aggregate formation in any given animal, we had observed striking variation in Q40 populations regarding the number of aggregates observed in individuals. Within a population of Q40 young adults, we observed animals with as few as 5 and as many as 140 aggregates despite similar expression levels of Q40-YFP protein, as determined by Western blotting of whole-animal extracts with antibody to green fluorescent protein followed by scanning densitometry. To address whether variation in Q40 aggregation phenotypes was due to a heritable factor, we examined whether the number of aggregates in the parent influenced the number of aggregates in the progeny. Because C. elegans can reproduce as a hermaphrodite, three individuals with fewer than 20 polyQ aggregates (Q40 “low”) and four animals with more than 80 aggregates (Q40 “high”) were allowed to lay eggs for 6 h, and aggregates were counted in the progeny. The average number of aggregates per animal after 3 days was similar whether the parent had few or many aggregates (Q40 low progeny=54±21, n=80; Q40 high progeny=54±21, n=100). These results suggest that substantial variation can exist at intermediate polyQ lengths even in a uniform genetic background. Other explanations for this polymorphism include polyQ repeat expansion or contraction; however, if the repeats were dramatically unstable we might have expected strains of Q19 or Q29 that exhibited aggregates or strains of Q40 that were no longer polymorphic. We have not observed any drift in these transgenic strains, which have been maintained in continuous culture for more than 2 years and retained their original phenotypes.

The polymorphism of aggregation phenotypes and motility defects in Q40 animals provided an opportunity to test whether the formation of aggregates was directly linked to cellular toxicity, which was accomplished by measuring motility and subsequently assessing, by fluorescence microscopy, the number of polyQ-YFP aggregates in the same animal. Q40 animals with the fewest aggregates exhibited a motility index that overlaps with that observed for the nontransgenic animals in the same population and separately with wild-type N2 animals, whereas Q40 animals with the largest number of aggregates exhibited a reduced motility similar to Q82 animals. Linear regression analysis resulted in an R2 value of −0.93, which reveals that greater than 90% of the variation in toxicity can be explained by the extent to which the protein has formed aggregates. Although these results provide evidence that formation of aggregates is correlated directly with toxicity, we cannot distinguish between aggregates themselves causing toxicity or a common mechanism leading to both aggregate formation and cellular dysfunction.

Aging-Dependent Shift in the Threshold for polyQ Aggregation and Toxicity. In further support for the existence of polymorphism at the threshold, we observed the appearance of protein aggregates as Q33 and Q35 animals aged (>4-5 days), which led us to perform an experiment in which individual Q0, Q29, Q33, Q35, Q40, and Q82 animals were examined daily for the appearance of protein aggregates and motility. Relative to Q40 and Q82 animals that quickly accumulated aggregates and exhibited a rapid decline in motility, Q33 and Q35 animals exhibited an initial lag before the gradual accumulation of aggregates to levels much lower, however, than for Q40 or Q82. For example, aging-dependent aggregate accumulation can be seen by comparison of the same Q35 animal at 4, 7, and 10 days. Q33 and Q35 animals also exhibited an age-dependent decline in motility. Q35-YFP fluorescence in young adults recovered rapidly after photobleaching, similar to that observed for Q0 or Q29 animals, whereas the Q35-YFP in older animals did not recover, consistent with conversion to the aggregated state. These results reveal that the threshold for polyQ aggregation and toxicity is not static. At three days of age or less, only animals expressing Q40 or greater exhibit aggregates. However, at 4-5 days of age the threshold shifts as aggregates appear in Q33 and Q35 animals. The threshold again shifts to Q29 in aged animals (>9-10 days). Thus, the threshold for polyQ aggregation is dynamic and likely reflects a balance of different factors including repeat length and changes in the cellular protein-folding environment over time.

Lifespan-Extending Mutation Delays the Onset of polyQ-Mediated Aggregation and Toxicity. Our results reveal that the threshold for polyQ aggregation and cytotoxicity in vivo is dynamic throughout the lifetime of an animal. The availability of C. elegans mutants with extended lifespans allowed us to test whether this dynamic behavior result from the intrinsic properties of a protein motif, or whether changes over time reflect the influence of aging-related alterations in the cell. We generated transgenic animals expressing Q82-YFP in the background of the age-1 (hx546) mutation or age-1 RNAi. age-1 encodes a phosphoinositide 3-kinase that functions in an insulin-like signaling pathway, and mutations in this gene can extend the lifespan (21, 27, 28). Q82-YFP in the age-1 (hx546) background (Q82; age-1) exhibited reduced aggregate formation in embryos relative to Q82-YFP in the wild-type background. Q82 aggregate formation was also reduced 30-50% during larval stages (1-2 days old) in age-1 animals compared with wild-type animals and was significantly lower until 4-5 days of age. Parallel motility assays also demonstrated a striking delay in onset of the motility defect, consistent with slower aggregate accumulation in Q82; age-1 animals.

To test whether loss of age-1 function would also influence aggregation and toxicity of other polyQ lengths, we subjected Q40 animals to age-1 RNAi. Both aggregation and onset of motility defects were delayed in Q40; age-1 (RNAi) animals. In wild-type animals, the kinase activity of AGE-1 is required in a signaling cascade that results in constitutive repression of the forkhead transcription factor DAF-16, leading to normal lifespan (22, 28, 29). Derepression of DAF-16 in age-1 animals results in an extended lifespan, and daf-16 mutations suppress the longevity phenotype (22, 29). To examine whether age-1 effects on longevity and polyQ aggregation and toxicity are mediated through similar regulatory pathways, we tested whether age-1 suppression of Q82 phenotypes was affected by inactivation of daf-16 by using RNAi. Q82;age-1;daf-16 animals exhibited aggregation and motility phenotypes similar to Q82 expressed in wild-type background, suggesting that lifespan extension and polyQ toxicity suppression mediated by age-1 share a common genetic pathway.

polyQ expansions are typically associated with neurodegenerative diseases in humans, yet the underlying principles of protein homeostasis and protein misfolding are universal properties of proteins in all cell types. Consistent with this premise is the appearance of polyQ-expansion protein aggregates in the yeast Saccharomyces cerevisiae and the expression of polyQ and α-synuclein protein aggregates in Drosophila (7, 30-32). Previous studies describing expression of polyQ-containing proteins in sensory neurons of C. elegans have demonstrated that numerous pathological features can be recapitulated (33, 34). The polyQ-length dependence of toxicity and variability among animals expressing near-threshold repeat lengths observed here suggests that, despite the obvious differences between C. elegans muscle cells and human neurons, the biochemical fates of polyQ proteins are indistinguishable. The demonstration that protein aggregation and toxicity are intensified during aging and the role of the age-1 mutation in suppressing these phenotypes highlight the utility of C. elegans as an animal model system to address these complex biochemical and behavioral phenotypes.

How might polyQ-initiated aggregates mediate the development of cellular toxicity in C. elegans? One explanation for aggregate-mediated toxicity results from observations that expanded polyQ tracts can sequester cellular proteins containing shorter polyQ domains, including transcription factors or coactivators such as CREB-binding protein (CBP) (35, 36). Recruitment of CBP into aggregates was shown to be associated with neuronal toxicity; moreover, reduced transcription from CBP-dependent genes was rescued by overexpression of CBP (36). Likewise, in C. elegans, we have shown that Q82 aggregates cause the relocalization of normally soluble Q19-YFP and a nuclear glutamine-rich splicing factor (HRP-1) into cytoplasmic aggregates (19). The predicted C. elegans proteome contains ≈200 proteins with polyQ or polar amino-acid-rich motifs, including the worm ortholog of CBP (37). It is not unreasonable, therefore, to suggest that some of these proteins are sequestered over time by the aggregates and have a role in polyQ-mediated toxicity in C. elegans. Another potential explanation for myocyte dysfunction could be disruption of the actin and myosin myofibrillar networks by polyQ aggregates. It is not clear whether the size or location of the aggregates are important in myocyte dysfunction. polyQ aggregate-mediated disruption of neurofilament networks has been observed in cultured neuroblasts and has been suggested to contribute to polyQ-mediated cellular toxicity (38).

The threshold hypothesis of polyQ-mediated cytotoxicity suggests that expansion of a glutamine homopolymer beyond a critical length results in a transition in the disposition or activities of the disease gene products. Consistent with this idea, in vitro studies on polyQ peptides of various lengths have demonstrated nucleation-dependent aggregation kinetics with a lag phase and rate of accumulation that depends on repeat length (39, 40). The lag period and rate of aggregate accumulation, however, are not linear. For example, synthetic peptides of Q44 exhibit a lag period of several hours followed by very rapid aggregate accumulation, peptides of Q37 and Q41 aggregate less rapidly after a lag period of approximately 20 h, and peptides of Q25-Q32 aggregate very slowly with lag times of up to 100 h (40). These results establish that the intrinsic properties of polyQ proteins are consistent with the inverse correlation between repeat length and age-of-onset observed in human polyQ diseases. However, what had not been addressed were the properties of polyQ proteins at threshold in the crowded macromolecular environment of the cell.

Our ability to monitor the biochemical properties of polyQ proteins in transparent C. elegans provided an opportunity to examine aggregation kinetics and its effects on cellular function throughout the lifespan of a living organism. The kinetics and length dependence of aggregation observed in living C. elegans exhibited striking similarity to those observed in vitro (39, 40). Animals expressing Q82 rapidly accumulated aggregates starting in embryos. Aggregates developed in Q40 animals at a similar rate, but with a delay of 1-2 days. Q33 and Q35 animals accumulated aggregates at a much slower rate and to smaller numbers only after a lag period of 4-5 days, and aggregates in Q29 animals appeared only after a week. For each polyQ length tested, the development of a motility defect paralleled the rate of aggregate accumulation. Taken together, these data suggest that the intrinsic parameters governing self-association of polyQ motifs derived from studies with synthetic peptides are manifest in living animals and may underlie the relationship between repeat length and age-of-onset in human polyQ diseases. However, patients with the same repeat length, especially near the threshold, can exhibit markedly different ages-of-onset and disease courses (9-12), suggesting that these intrinsic parameters interact with modifiers of pathology.

The identification of age-1 as a genetic modifier of protein aggregation is intriguing, and one interpretation of these results is that age-1 and daf-16 define a genetic pathway that governs aging and may do so by influencing the biochemical events that that have an impact on protein homeostasis as monitored by the appearance of protein aggregates. For example, daf-16 could be a regulator of chaperone or proteasome activity or indirectly have effects by changes in metabolism influencing the overall synthesis or degradation rate of proteins. Insulin-like signaling in C. elegans regulates not only longevity but also entry into the alternative developmental state of dauer diapause (21, 22, 27-29). Entry into the dauer state results in elevated levels of molecular chaperones, such as Hsp70 and Hsp90 (41, 42). However, sequence analysis of the promoter regions from some particular Hsp16, Hsp70, and Hsp90 homologs in the C. elegans genome did not reveal the presence of the previously characterized daf-16 family binding element TTGTTTAC (43), arguing against direct transcriptional regulation of these particular chaperone genes by DAF-16.

Loss of age-1 function is pleiotropic, and animals bearing this mutation are resistant to a variety of environmental stresses, including heat shock and reactive oxygen species (44-46). No precedent exists for direct effects of insulin-like signaling on stress-response regulators, such as the heat-shock transcription factor. However, previous studies have shown that the heat-shock response is induced poorly during aging as a result of reduced heat-shock transcription factor activity (47, 48). Consequently, the ability of chaperone networks to respond to the appearance of misfolded and aggregation-prone proteins during aging would be compromised, which is consistent with forward and reverse genetic approaches that have identified molecular chaperones as suppressors in models of protein aggregation-related diseases (49-52).

The delay in onset of polyQ-associated phenotypes observed in age-1 animals implies that the rate of progression for proteinopathies is linked with the genetic regulation of aging. This finding, although unexpected, is supported by observations that the time until polyQ-mediated pathology develops (days in C. elegans, weeks in Drosophila, months in mice, and years in humans) correlates approximately with the lifespan of the organism. This link suggests that strategies to extend lifespan could also delay the onset of aging-related diseases characterized by the appearance of misfolded and aggregation-prone proteins.

We observed genetic interactions between the protein hsf1 (heat shock factor 1) and the age1/daf2 longevity pathway. These observations indicate that these pathways are genetically linked. Moreover, changes in the levels of hsf1 alters protein aggregation. Overexpression of hsf1 results in the suppression of polyglutamine aggregation and suppression of hsf sensitizes animals to premature polyglutamine.
1. Kopito, R. R. & Ron, D. (2000) Nat. Cell Biol. 2, E207-E209.
2. Horwich, A. L. & Weissman, J. S. (1997) Cell 89, 499-510.
3. Perutz, M. F. (1999) Trends Biochem. Sci. 24, 58-63.
4. Orr, H. T. (2001) Genes Dev. 15, 925-932.
5. Mangiarini, L., Sathasivam, K., Seller, M., Cozens, B., Harper, A., Hetherington, C., Lawton, M., Trottier, Y., Lehrach, H., Davies, S. W. & Bates, G. P. (1996) Cell 87, 493-506.
6. Ordway, J. M., Tallaksen-Greene, S., Gutekunst, C. A., Bernstein, E. M., Cearley, J. A., Wiener, H. W., Dure, L. S., IV, Lindsey, R., Hersch, S. M., Jope, R. S., et al. (1997) Cell 91, 753-763.
7. Warrick, J. M., Paulson, H. L., Gray-Board, G. L., Bui, Q. T., Fischbeck, K. H., Pittman, R. N. & Bonini, N. M. (1998) Cell 93, 939-949.
8. The Huntington's Disease Collaborative Research Group. (1993) Cell 72, 971-983.
9. Duyao, M., Ambrose, C., Myers, R., Novelletto, A., Persichetti, F., Frontali, M., Folstein, S., Ross, C., Franz, M., Abbott, M., et al. (1993) Nat. Genet. 4, 387-392.
10. Snell, R. G., MacMillan, J. C., Cheadle, J. P., Fenton, I., Lazarou, L. P., Davies, P., MacDonald, M. E., Gusella, J. F., Harper, P. S. & Shaw, D. J. (1993) Nat. Genet. 4, 393-397.
11. Andrew, S. E., Goldberg, Y. P., Kremer, B., Telenius, H., Theilmann, J., Adam, S., Starr, E., Squitieri, F., Lin, B., Kalchman, M. A., et al. (1993) Nat. Genet. 4, 398-403.
12. Brinkman, R. R, Mezei, M. M., Theilmann, J., Almqvist, E. & Hayden, M. R. (1997) Am. J. Hum. Genet. 60, 1202-1210.
13. Davies, S. W., Turmaine, M., Cozens, B. A., DiFiglia, M., Sharp, A. H., Ross, C. A., Scherzinger, E., Wanker, E. E., Mangiarini, L. & Bates, G. P. (1997) Cell 90, 537-548.
14. Paulson, H. L. (1999) Am. J. Hum. Genet. 64, 339-345.
15. Klement, I. A., Skinner, P. J., Kaytor, M. D., Yi, H., Hersch, S. M., Clark, H. B., Zoghbi, H. Y. & Orr, H. T. (1998) Cell 95, 41-53.
16. Saudou, F., Finkbeiner, S., Devys, D. & Greenberg, M. E. (1998) Cell 95, 55-66.
17. Simeoni, S., Mancini, M. A., Stenoien, D. L., Marcelli, M., Weigel, N. L., Zanisi, M., Martini, L. & Poletti, A. (2000) Hum. Mol. Genet. 9, 133-144.
18. Huynh, D. P., Figueroa, K., Hoang, N. & Pulst, S. M. (2000) Nat. Genet. 26, 44-50.
19. Satyal, S. H., Schmidt, E., Kitagawa, K., Sondheimer, N., Lindquist, S., Kramer, J. M. & Morimoto, R. I. (2000) Proc. Natl. Acad. Sci. USA 97, 5750-5755.
20. Fire, A., Harrison, S. W. & Dixon, D. (1990) Gene 93, 189-198.
21. Morris, J. Z., Tissenbaum, H. A. & Ruvkun, G. (1996) Nature (London) 382, 536-539.
22. Lin, K., Dorman, J. B., Rodan, A. & Kenyon, C. (1997) Science 278, 1319-1322.
23. Timmons, L., Court, D. L. & Fire, A. (2001) Gene 263, 103-112.
24. Brenner, S. (1974) Genetics 77, 71-94.
25. Lewis, J. A. & Fleming, J. T. (1995) in Caenorhabditis elegans: Modern Biological Analysis of an Organism, eds. Epstein, H. F. & Shakes, D. C. (Academic, San Diego), pp. 4-29.
26. Swaminathan, R., Hoang, C. P. & Verkman, A. S. (1997) Biophys. J. 72, 1900-1907.
27. Friedman, D. B. & Johnson, T. E. (1988) Genetics 118, 75-86.
28. Tissenbaum, H. A. & Ruvkun, G. (1998) Genetics 148, 703-717.
29. Ogg, S., Paradis, S., Gottlieb, S., Patterson, G. I., Lee, L., Tissenbaum, H. A. & Ruvkun, G. (1997) Nature (London) 389, 994-999.
30. Feany, M. B. & Bender, W. W. (2000) Nature (London) 404, 394-398.
31. Krobitsch, S. & Lindquist, S. (2000) Proc. Natl. Acad. Sci. USA 97, 1589-1594.
32. Marsh, J. L., Walker, H., Theisen, H., Zhu, Y. Z., Fielder, T., Purcell, J. & Thompson, L. M. (2000) Hum. Mol. Genet. 9, 13-25.
33. Faber, P. W., Alter, J. R., MacDonald, M. E. & Hart, A. C. (1999) Proc. Natl. Acad. Sci. USA 96, 179-184.
34. Parker, J. A., Connolly, J. B., Wellington, C., Hayden, M., Dausset, J. & Neri, C. (2001) Proc. Natl. Acad. Sci. USA 98, 13318-13323.
35. Steffan, J. S., Kazantsev, A., Spasic-Boskovic, 0., Greenwald, M., Zhu, Y. Z., Gohler, H., Wanker, E. E., Bates, G. P., Housman, D. E. & Thompson, L. M. (2000) Proc. Natl. Acad. Sci. USA 97, 6763-6768.
36. Nucifora, F. C., Jr., Sasaki, M., Peters, M. F., Huang, H., Cooper, J. K., Yamada, M., Takahashi, H., Tsuji, S., Troncoso, J., Dawson, V. L., et al. (2001) Science 291, 2423-2428.
37. Michelitsch, M. D. & Weissman, J. S. (2000) Proc. Natl. Acad. Sci. USA 97, 11910-11915.
38. Nagai, Y., Onodera, O., Chun, J., Strittmatter, W. J. & Burke, J. R. (1999) Exp. Neurol. 155, 195-203.
39. Scherzinger, E., Sittler, A., Schweiger, K., Heiser, V., Lurz, R., Hasenbank, R., Bates, G. P., Lehrach, H. & Wanker, E. E. (1999) Proc. Natl. Acad. Sci. USA 96, 4604-4669.
40. Chen, S., Berthelier, V., Yang, W. & Wetzel, R. (2001) J. Mol. Biol. 311, 173-182.
41. Dalley, B. K. & Golomb, M. (1992) Dev. Biol. 151, 80-90.
42. Cherkasova, V., Ayyadevara, S., Egilmez, N. & Reis, R. S. (2000) J. Mol. Biol. 300, 433-448.
43. Furuyama, T., Nakazawa, T., Nakano, I. & Mori, N. (2000) Biochem. J. 349, 629-634.
44. Lithgow, G. J., White, T. M., Melov, S. & Johnson, T. E. (1995) Proc. Natl. Acad. Sci. USA 92, 7540-7544.
45. Larsen, P. L. (1993) Proc. Natl. Acad. Sci. USA 90, 8905-8509.
46. Kenyon, C. (2001) Cell 105, 165-168.
47. Fargnoli, J., Kunisada, T., Fornace, A. J., Jr., Schneider, E. L. & Holbrook, N. J. (1990) Proc. Natl. Acad. Sci. USA 87, 846-840.
48. Fawcett, T. W., Sylvester, S. L., Sarge, K. D., Morimoto, R. I. & Holbrook, N. J. (1994) J. Biol. Chem. 269, 32272-32278.
49. Cummings, C. J., Mancini, M. A., Antalffy, B., DeFranco, D. B., Orr, H. T. & Zoghbi, H. Y. (1998) Nat. Genet. 19, 148-154.
50. Warrick, J. M., Chan, H. Y., Gray-Board, G. L., Chai, Y., Paulson, H. L. & Bonini, N. M. (1999) Nat. Genet. 23, 425-428.
51. Fernandez-Funez, P., Nino-Rosales, M. L., de Gouyon, B., She, W. C., Luchak, J. M., Martinez, P., Turiegano, E., Benito, J., Capovilla, M., Skinner, P. J., et al. (2000) Nature (London) 408, 101-106.
52. Auluck, P. K., Chan, H. Y., Trojanowski, J. Q., Lee, V. M. & Bonini, N. M. (2002) Science 295, 865-868.

A number of embodiments of the inventions have been described. Nevertheless, it will be understood that various modifications may be made without departing from the spirit and scope of the inventions. Accordingly, other embodiments are within the scope of the following claims.

What is claimed:

1. A method of treating or preventing Huntington's Disease in a subject, the method comprising: reducing activity of the IGF-1/GH axis in a subject who has or is at risk of having Huntington's disease.

2. The method of claim 1, wherein the subject is human.

3. The method of claim 1, that comprises administering a composition that reduces IGF-1/GH axis activity.

4. The method of claim 3, wherein the composition is administered in an amount effective to reduce or prevent Huntington's Disease.

5. The method of claim 3, wherein the composition is an agonist of an inhibitory component of the IGF-1/GH axis.

6. The method of claim 3, wherein the composition is an antagonist of an activator of the IGF-1/GH axis or a component of the IGF-1/GH axis.

7. The method of claim 5, wherein the inhibitory component of the IGF-1/GH axis is a somatostatin receptor, a PTEN transcription factor, or a FOXO transcription factor.

8. The method of claim 5, wherein the agonist is somatostatin, L-054,522, BIM-23244, BIM-23197, BIM-23268, octreotide, TT-232, butreotide, lanreotide, or vapreotide.

9. The method of claim 6, wherein the component of the IGF-1/GH axis is GH, GHRH, GHRH-R, GHS, GHS-R, GH-R, PI-3 kinase, PDK-1, or an AKT kinase.

10. The method of claim 9, wherein the antagonist is a kinase inhibitor.

11. The method of claim 9, wherein the antagonist is an antibody to a hormone or an antibody to a cell surface receptor, or functional fragment thereof.

12. The method of claim 9, wherein the antagonist binds to a cell surface receptor.

13. The method of claim 12, wherein the antagonist is a modified ligand of the cell surface receptor.

14. The method of claim 9, wherein the antagonist is a modified growth hormone molecule that antagonizes GH-R.

15. The method of claim 9, wherein the antagonist is Pegvisomant.

16. The method of claim 3, wherein the compound is a dopamine agonist that decreases GH production.

17. The method of claim 3, wherein the treatment is commenced at least prior to clinical onset of Huntington's disease.

18. The method of claim 3, wherein the treatment is provided at least at some point after clinical onset of Huntington's disease.

19. A method of evaluating a compound for ability to modulate Huntington's disease-related polyglutamine aggregation in a cell, the method comprising:
a) providing a test compound;
b) contacting the test compound to a GH/IGF-1 axis component in vitro;
c) evaluating interaction between the test compound and the growth hormone/IGF-1 axis component;
d) contacting the test compound to a cell; and
e) evaluating polyglutamine aggregation in or around the cell or evaluating the cell for a cellular symptom of polyglutamine aggregation.

20. A method for evaluating compounds for ability to modulate Huntington's disease-related polyglutamine aggregation in an organism, the method comprising:
a) providing a library of compounds;
b) contacting each compound of the library to a GH/IGF-1 axis component in vitro;
c) evaluating interaction between each compound and the GH/IGF-1 axis component;
d) selecting a subset of compounds from the library based on the evaluated interactions; and
e) for each compound of the subset, contacting the compound to a cell, and evaluating polyglutamine aggregation in or around the cell or evaluating the cell for a cellular symptom of polyglutamine aggregation.

21. A method of evaluating a compound for ability to modulate Huntington's disease-related polyglutamine aggregation in an organism, the method comprising:
a) providing a test compound;
b) contacting the test compound to a GH/IGF-1 axis component in vitro;
c) evaluating interaction between the test compound and the growth hormone/IGF-1 axis component;
d) administering the test compound to a subject organism; and
e) evaluating the subject organism for polyglutamine aggregation, a symptom of polyglutamine aggregation, or a neurological symptom.

22. A method for evaluating compounds for ability to modulate Huntington's disease-related polyglutamine aggregation in an organism, the method comprising:
a) providing a library of compounds;
b) contacting each compound of the library to a GH/IGF-1 axis component in vitro;
c) evaluating interaction between each compound and the GH/IGF-1 axis component;
d) selecting a subset of compounds from the library based on the evaluated interactions; and
e) for each compound of the subset, administering the compound to a subject organism, and evaluating the subject organism for polyglutamine aggregation, a symptom of polyglutamine aggregation, or a neurological symptom.

23. The method of claim 19, wherein the cell expresses a heterologous protein that includes a polyglutamine repeat that includes at least 35 glutamines.

24. The method of claim 19, wherein the cell expresses an endogenous protein that includes a polyglutamine repeat that includes at least 35 glutamines.

25. The method of claim 23, wherein the heterologous protein comprises at least 50 amino acids of the amino acid sequence of exon 1 of the Huntingtin protein.

26. The method of claim 19, wherein the evaluating comprises photobleaching and evaluating recovery of fluorescence after photobleaching.

27. A cultured cell preparation comprising:
an engineered mammalian cell that expresses a protein that comprises at least 50 amino acid of exon 1 of the Huntingtin protein and a polyglutamine repeat region of at least 35 glutamines; and
medium containing an agonist of the GH/IGF-1 axis.

28. A method for evaluating a test compound, the method comprising:
providing the cultured cell preparation of claim 27;
contacting a test compound to cells in the preparation; and
evaluating the cells for aggregation of the protein with the polyglutamine repeats or a symptom of Huntington's disease.
